SEQUENCE LISTING (14 sequences)

SEQ ID NO: 1 - protein, 154 aa, Rattus norvegicus
  REGION 1..154: note = Rat myoglobin
MGLSDGEWQMVLNIWGKVEGDLAGHGQEVLISLFKAHPETLEKFDKFKNLKSEEEMKSSEDLKKHGCTVLTALGTILKKKGQHAAEIQPLAQSHATKHKIPVKYLEFISEVIIQVLKKRYSGDFGADAQGAMSKALELFRNDIAAKYKELGFQG

SEQ ID NO: 2 - protein, 154 aa, Oryctolagus cuniculus
  REGION 1..154: note = Rabbit myoglobin
MGLSDAEWQLVLNVWGKVEADLAGHGQEVLIRLFHTHPETLEKFDKFKHLKSEDEMKASEDLKKHGNTVLTALGAILKKKGHHEAEIKPLAQSHATKHKIPVKYLEFISEAIIHVLHSKHPGDFGADAQAAMSKALELFRNDIAAQYKELGFQG

SEQ ID NO: 3 - protein, 154 aa, Mus musculus
  REGION 1..154: note = Mouse myoglobin
MGLSDGEWQLVLNVWGKVEADLAGHGQEVLIGLFKTHPETLDKFDKFKNLKSEEDMKGSEDLKKHGCTVLTALGTILKKKGQHAAEIQPLAQSHATKHKIPVKYLEFISEIIIEVLKKRHSGDFGADAQGAMSKALELFRNDIAAKYKELGFQG

SEQ ID NO: 4 - protein, 154 aa, Mammuthus trogontherii
  REGION 1..154: note = Steppe mammoth myoglobin
MGLSDGEWELVLKTWGKVEADIPGHGLEVFVRLFTGHPETLEKFDKFKHLKTEGEMKASEDLKKQGVTVLTALGGILKKKGHHQAEIQPLAHSHATKHKIPIKYLEFISDAIIHVLQSKHPAEFGADAQGAMKKALELFRNDIAAKYKELGFQG

SEQ ID NO: 5 - protein, 154 aa, Mammuthus primigenius
  REGION 1..154: note = Woolly mammoth myoglobin
MGLSDGEWELVLKTWGKVEADIPGHGLEVFVRLFTGHPETLEKFDKFKHLKTEGEMKASEDLKKQGVTVLTALGGILKKKGHHQAEIQPLAQSHATKHKIPIKYLEFISDAIIHVLQSKHPAEFGADAQGAMKKALELFRNDIAAKYKELGFQG

SEQ ID NO: 6 - protein, 147 aa, Thunnus orientalis
  REGION 1..147: note = Tuna myoglobin
MADFDAVLKCWGPVEADYTTIGGLVLTRLFKEHPETQKLFPKFAGIAQADIAGNAAVSAHGATVLKKLGELLKAKGSHAAILKPLANSHATKHKIPINNFKLISEVLVKVMHEKAGLDAGGQTALRNVMGIIIADLEANYKELGFSG

SEQ ID NO: 7 - DNA, 462 bp, synthetic construct
  misc_feature 1..462: note = Rat myoglobin coding nucleid acid
  source 1..462: mol_type = other DNA
atgggcctgagcgatggcgaatggcagatggtgctgaacatttggggcaaagtggaaggcgatctggcgggccatggccaggaagtgctgattagcctgtttaaagcgcatccggaaaccctggaaaaatttgataaatttaaaaacctgaaaagcgaagaagaaatgaaaagcagcgaagatctgaaaaaacatggctgcaccgtgctgaccgcgctgggcaccattctgaaaaaaaaaggccagcatgcggcggaaattcagccgctggcgcagagccatgcgaccaaacataaaattccggtgaaatatctggaatttattagcgaagtgattattcaggtgctgaaaaaacgctatagcggcgattttggcgcggatgcgcagggcgcgatgagcaaagcgctggaactgtttcgcaacgatattgcggcgaaatataaagaactgggctttcagggc

SEQ ID NO: 8 - DNA, 462 bp, synthetic construct
  misc_feature 1..462: note = Rabbit myoglobin coding nucleid acid
  source 1..462: mol_type = other DNA
atgggcctgagcgatgcggaatggcagctggtgctgaacgtgtggggcaaagtggaagcggatctggcgggccatggccaggaagtgctgattcgcctgtttcatacccatccggaaaccctggaaaaatttgataaatttaaacatctgaaaagcgaagatgaaatgaaagcgagcgaagatctgaaaaaacatggcaacaccgtgctgaccgcgctgggcgcgattctgaaaaaaaaaggccatcatgaagcggaaattaaaccgctggcgcagagccatgcgaccaaacataaaattccggtgaaatatctggaatttattagcgaagcgattattcatgtgctgcatagcaaacatccgggcgattttggcgcggatgcgcaggcggcgatgagcaaagcgctggaactgtttcgcaacgatattgcggcgcagtataaagaactgggctttcagggc

SEQ ID NO: 9 - DNA, 462 bp, synthetic construct
  misc_feature 1..462: note = Mouse myoglobin coding nucleid acid
  source 1..462: mol_type = other DNA
atgggcctgagcgatggcgaatggcagctggtgctgaacgtgtggggcaaagtggaagcggatctggcgggccatggccaggaagtgctgattggcctgtttaaaacccatccggaaaccctggataaatttgataaatttaaaaacctgaaaagcgaagaagatatgaaaggcagcgaagatctgaaaaaacatggctgcaccgtgctgaccgcgctgggcaccattctgaaaaaaaaaggccagcatgcggcggaaattcagccgctggcgcagagccatgcgaccaaacataaaattccggtgaaatatctggaatttattagcgaaattattattgaagtgctgaaaaaacgccatagcggcgattttggcgcggatgcgcagggcgcgatgagcaaagcgctggaactgtttcgcaacgatattgcggcgaaatataaagaactgggctttcagggc

SEQ ID NO: 10 - DNA, 465 bp, synthetic construct
  misc_feature 1..465: note = Steppe mammoth myoglobin coding nucleid acid
  source 1..465: mol_type = other DNA
atgggactcagcgacggggaatgggagttggtgttgaaaacctgggggaaagtggaggctgacatcccgggccatgggctggaagtcttcgtcaggctcttcacaggtcatcccgagaccctggagaagttcgacaagtttaagcacctgaagacagagggcgagatgaaggcctccgaggacctgaagaagcaaggtgttactgtgctcactgccctggggggcatcctcaagaagaaagggcatcaccaggcggagattcagcccctggcccattctcatgccaccaagcacaagatccccatcaagtatctggagttcatctcggacgccatcatccacgttctgcaaagcaagcatcctgcggaatttggcgctgatgcccagggagccatgaaaaaggccttggagctgttccggaatgacattgcggccaagtataaggagctggggttccagggctag

SEQ ID NO: 11 - DNA, 465 bp, synthetic construct
  misc_feature 1..465: note = Woolly mammoth myoglobin coding nucleid acid
  source 1..465: mol_type = other DNA
atgggactcagcgacggggaatgggagttggtgttgaaaacctgggggaaagtggaggctgacatcccgggccatgggctggaagtcttcgtcaggctcttcacaggtcatcccgagaccctggagaagttcgacaagtttaagcacctgaagacagagggcgagatgaaggcctccgaggacctgaagaagcaaggtgttactgtgctcactgccctggggggcatcctcaagaagaaagggcatcaccaggcggagattcagcccctggcccagtctcatgccaccaagcacaagatccccatcaagtatctggagttcatctcggacgccatcatccacgttctgcaaagcaagcatcctgcggaatttggcgctgatgcccagggagccatgaaaaaggccttggagctgttccggaatgacattgcggccaagtataaggagctggggttccagggctag

SEQ ID NO: 12 - DNA, 444 bp, synthetic construct
  misc_feature 1..444: note = Tuna myoglobin coding nucleid acid
  source 1..444: mol_type = other DNA
atggctgactttgatgcagttctgaagtgttggggtccagtggaggcggactacaccaccattggaggcctggttctgacccgtttattcaaagagcaccctgagacccagaagctgttccccaaattcgctggcatcgcccaggctgacatagccggtaacgcagctgtttctgctcatggtgccactgtgctgaagaaacttggagagctgctgaaggccaaaggcagtcacgctgccatcctaaaaccactggcaaacagccatgccactaagcacaagattcccattaataacttcaagctgatttctgaggtccttgtgaaggtcatgcatgagaaggcaggactcgatgccggtgggcagacagccctgaggaacgtgatgggtatcatcatcgccgaccttgaggccaactacaaagagctgggcttctctggctga

SEQ ID NO: 13 - protein, 175 aa, Mammuthus primigenius
  REGION 1..175: note = Woolly mammoth k-casein
MKGFLLVVNILLLPLFLAAEVQNQEESRCLEKDERWFCQKAVKYIPNDYVLKSYYRYEPNYNQFRAAVPINNPYLIYLYPAKQVAVRPHTQIQWQVPSNIYPSPSVPHTYLKPPFIIPPKKTQDKPIIPPTGTVASIEATVEPKVNTVVNAEASSEFIATNTPEATTVPVISPQI

SEQ ID NO: 14 - protein, 260 aa, Mammuthus primigenius
  REGION 1..260: note = Woolly mammoth b-casein
MKVFILACLVAFALGRETVENLSSSEIRQFYSEQKPEGVKHEEQQREDEHQNKIQPLFQPQPLVYPFAEPIPYTVFPPNAIPLAQPIVVLPFPQPEVQLPEAKEITFPRQKLMSFLKSPVMPFFDPQPNLGTDLENLHLPLPLLQPLRHQLHQPLAQTPVLPLPLSLPKVLPVPQQVIPYPQRGRPIQNLLYEEPLLDPTRKIYPVAQPLAPVYNPVAYMIGIPCCSTLLTYLHQSSRSQYPIQNKLGYLIAMPKKVRPT

(19) United States
(12) Patent Application Publication
(10) Pub. No.: US 2026/0240201 A1
(43) Pub. Date: Aug. 20, 2026

(54) Method for preparing a pet food

(71) Applicant: Paleo B.V., Diest (BE)

(72) Inventors: Andy De Jong, Diest (BE); Hermes Sanctorum, Diest (BE); Elsa Lauwers, Diest (BE)

(73) Assignee: Paleo B.V., Diest (BE)

(21) Appl. No.: 18/876,670

(22) PCT Filed: Jun. 28, 2023
(86) PCT No.: PCT/EP2023/067608

§ 371 Date: Dec. 19, 2024

(30) Foreign Application Priority Data
Jun. 29, 2022 (EP) 22181886.7

(51) Int. Cl. A23K 20/147; A23K 10/10; A23K 40/25; A23K 50/42; A23K 50/45; A23K 50/48; C07K 14/805; C12N 15/81; C12R 1/84

(52) U.S. Cl. CPC A23K 20/147; A23K 10/10; A23K 40/25; A23K 50/42; A23K 50/45; A23K 50/48; C07K 14/805; C12N 15/815; C12R 2001/84

(57) ABSTRACT

Described herein a method for preparing a pet food, wherein the method comprises adding an isolated myoglobin, wherein the isolated myoglobin is a rat myoglobin, a rabbit myoglobin, a mouse myoglobin, a mammoth myoglobin or a tuna myoglobin.

### FIELD

This inventions relates to a method for preparing a pet food, wherein the method comprises adding an isolated myoglobin, wherein the isolated myoglobin is a rat myoglobin, a rabbit myoglobin, a mouse myoglobin, a mammoth myoglobin or a tuna myoglobin.

### BACKGROUND

The increasing knowledge about pet nutrition and food technology has enabled the industry to develop and diversify in terms of the type, taste, composition, shape of pet food. However, commercial pet food is highly processed, meaning the nutritional value together with the taste and texture may be lost during industrial production.

Against this background, there is an ongoing strive to develop meat substitutes as pet foods to lower their ecological footprint. For these types of pet foods, obtaining a taste and texture close to that of unprocessed meat is hard, yet detrimental to obtain a threshold level of food acceptance by the animals.

Hence, there is an ongoing need in the art for pet foods that mimick certain aspects, such as taste and texture, of meat, while being environmentally friendly.

### DESCRIPTION

### Method for Preparing a Pet Food

In the first aspect of this invention, there is provided a method for preparing a pet food, wherein the method comprises adding an isolated myoglobin, wherein said isolated myoglobin is a rat myoglobin, a rabbit myoglobin, a mouse myoglobin, a mammoth myoglobin or a tuna myoglobin. Example 3 provides an example of such a method.

In an embodiment, the pet is a vertebrate. In an embodiment, the pet is a mammal, a bird, a fish, an amphibian, or a reptile. In an embodiment, the pet is a mammal. Mammals are a group of vertebrates constituting the class mammalia, characterized by the presence of mammary glands which in females produce milk for feeding (nursing) their young, a neocortex (a region of the brain), fur or hair, and three middle ear bones. In an embodiment, the pet is a carnivore or an omnivore, preferably a mammal. In a preferred embodiment, the pet may be a dog, a cat, a ferret, a fox, a bear, a wolf, a jaguar, a lion, a tiger, a snake, a piranha, or an alligator. In a preferred embodiment, the pet may be a dog or a cat.

In an embodiment, a rat is of any species in the genus Rattus. In more preferred embodiment, a rat is a black rat (Rattus rattus) or a bown rat (Rattus norvegicus). Different species in the genus Rattus may share the same myoglobin sequence.

In an embodiment, the pet food is a dry pet food, a semi-moist pet food, a wet pet food, or any combination of these types of pet food. A pet food may be formulated as a liquid, solid, or semi-solid pet food. By “dry pet food” is meant one that has a moisture content less than 15 percent by weight. By “semi-moist pet food” is meant one that has a moisture content between 55 to 65 percent by weight. By “wet type food” is meant one that has a moisture content between 65 to 85 percent by weight. Hereinafter, all percentages referred to are understood to be by weight unless specified otherwise and are based upon the weight of the final pet food product. Suitable examples of solid pet foods include kibbles, snacks, cell-based meats and meat substitutes, cultured meats and meat and plant-based meats meat substitutes. Suitable examples of snacks include a protein bar, a dental snack, meat cubes or chew sticks. Such plant-based meats may comprise plant proteins such as soy proteins. Suitable examples of semi-moist and wet type pet foods include pate or loaf, chunks in gravy or jelly or soup. In an embodiment, the pet food is a pate or loaf, or chunks in gravy.

In an embodiment, the pet food is a meat substitute. A meat substitute is per definition not meat, not natural meat, not genuine meat. A meat substitute may be considered as a non-naturally occurring food or edible product. It means that within the context of this invention, a meat substitute is not meat from or derived from rat, rabbit, mouse or tuna (or from or derived from mammoth if this animal would still exist which is not the case). A meat substitute is synonymous of a meat replica or a meat analogue product or a meat-like product. It is thus understood that cell-based meats, cultured meats and plant-based meats may be considered meat substitutes. Cell-based meat is normally cultivated directly from animal stem cells. Cultured meat is normally produced by in vitro cell cultures of animal cells, as a form of cellular agriculture. Plant-based meat is normally produced using plant and other non-animal products.

In an embodiment, the pet food is a meat substitute, wherein the meat substitute is a cell-based meat substitute, a cultured meat substitute or a plant-based meat substitute. Meat substitutes may be dry pet foods, semi-moist pet foods, wet pet foods or any combination of these types of pet foods. The inventors discovered that “meat experience” of a pet food comprising meat could be mimicked in a meat substitute by adding an isolated myoglobin as defined herein. Depending on the meat substitute envisaged, the skilled person would know which formulations are the most suitable.

In an embodiment, the pet food is a meat substitute, wherein the meat substitute mimicks the aspect (such as pattern, lettering), form, structure, composition (such as similar fat, protein and/or heme iron content), palatability, flavor, texture, color, aroma, appearance and/or nutritional value of meat. The degree of acceptance, the general liking or preference of one pet food over another, or the palatability of a pet food may be assessed by a preference/acceptance test. Palatability of pet foods is typically measured using a single-bowl or a two-bowl test or an operant lever-press test. For example, in a preference test, pets are offered a control meal and the test meal in a controlled environment. Experimenters record the rate at which the animals choose the test meal over the control meal. Larger tests can be conducted in a home setting by pet owners who are asked to record “food preference” behaviors such as time spent sniffing the food, licking lips or nose, ear or tail flicking, etc. This is mostly due to the addition of the isolated myoglobin as described herein. The meat substitute may not have all the drawbacks of meat. For example, the meat substitute may be healthier than meat. Moreover, the impact of the meat substitute on the environment (long term direct/indirect effect on climate change) is expected to be less detrimental than the known impact of meat.

Regarding nutritional value, Example 4 shows that the iron bioavailability of the pet food is higher than corresponding pet foods supplemented with non-heme iron. Example 5 details the color and color stability of the pet food. It is shown that the addition of myoglobin in a meat substitute either stabilizes and/or improves its desired color profile over time during cold storage, regardless of the raw or cooked state of the product. Example 6 reports the change in aromatic profile of a meat substitute upon addition of myoglobin, indicating that the levels of volatile compounds associated with meaty flavors are significantly increased in heat processed products that contain myoglobin.

In an embodiment, the pet food is a meat substitute, wherein the meat substitute is expected to mimick the composition (such as similar fat, protein and/or heme iron content), palatability, flavor, color and/or aroma of meat without having all its drawbacks. In an embodiment, the meat substitute is expected to mimick the flavor and/or aroma of meat without having all its drawbacks. In an embodiment, the meat substitute is expected to mimick the palatability of meat without having all its drawbacks. In an embodiment, the meat substitute is expected to mimick the flavor of meat without having all its drawbacks. In an embodiment, the meat substitute is expected to mimick the aroma of meat without having all its drawbacks. In the embodiments of this context, this is mostly due to the presence of the isolated myoglobin as described herein. Alternatively, it might have an aspect, form, structure, texture, color, palatability, flavor, aroma and/or appearance which is distinct from the one of meat.

Within the context of the invention, the meat experience may be mimicked when the meat substitute generates an aroma and/or a flavor compound recognizable by pets and characteristic of some odorants and/or flavor compound released when meat is cooked. For example, the formation of some of these odorants may be catalyzed by the iron of heme present in the isolated myoglobin. Without being bound to this theory, the formation may be due to lipid oxidation and/or Maillard reactions (“Maillard products”). This situation may be mimicked with the isolated myoglobin within the meat substitute of the invention.

In an embodiment of the invention, wherein the pet food is a meat substitute, preferably a raw meat substitute, wherein said raw meat substitute is expected to mimick the bloody and/or metallic aroma of meat. Preferably, the meat substitute is able to mimick the bloody and/or metallic aroma of meat to a higher degree than other meat substitutes (i.e. not according to the invention). In the context of this invention, “raw” means not subjected to a heat treatment, preferably uncooked and ungrilled.

In an embodiment of the invention, wherein the pet food is a meat substitute, preferably a cooked meat substitute, wherein said cooked meat substitute is expected to mimick the roasted aroma of meat, particularly of grilled meat. Preferably, the meat substitute is able to mimick the roasted aroma of meat to a higher degree than other meat substitutes (i.e. not according to the invention). In the context of this invention, “cooked” means subjected to a heat treatment, preferably sterilized, grilled or roasted.

In the context of this invention, and specifically in the embodiments in this section, an “other meat substitute” or “a meat substitute not according to the invention” is preferably a plant-based meat substitute comprising a recombinant soy leghemoglobin (LegH).

In an embodiment, the pet food has an aroma characterized by a higher concentration of Mailard compounds, i.e. compounds formed due to the Maillard reaction, when cooked, compared to a corresponding pet food which to which no isolated myoglobin has been added.

In an embodiment, the pet food, preferably suitable for dogs, has an aroma characterized by a higher concentration of 3-methylbutanal, 2-methyl-butanal, pyrazine, pyrrole, methylpyrazine, 2-ethyl-6-methyl-pyrazine, methional, 2,6-dimethylpyrazine, 2-acetylthiazole, 2,5-dimethylpyrazine, octanal and/or nonanal, when cooked, compared to a corresponding pet food which to which no isolated myoglobin has been added.

In an embodiment, the meat substitute, preferably a grilled meat substitute of the invention, has an aroma characterized by a higher concentration of oxidized lipids, lipid oxidation products, pyrazines, pyrroles, aldehydes and/or ketones in the volatile compounds obtained from the (grilled) meat substitute relative to other meat substitutes (i.e. not according to the invention). Higher preferably means at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or 300%. Preferred pyrazines are methylpyrazine, 2,6-dimethylpyrazine, 2,5-dimethylpyrazine, and 2-ethyl-6-methylpyrazine. A preferred pyrrole is pyrrole. Preferred lipid oxidation products are 2-methylbutanal and 3-methylbutanal. Preferred aldehydes are methional, nonanal and octanal. A preferred ketone is 2-acetylthiazole. It is understood that the categories of compounds mentioned in this paragraph are not mutually exclusive, e.g. lipid oxidation products may be aldehydes or ketones.

Aromas from meat substitutes containing different concentrations of recombinant myoglobin may be analyzed using gas chromatography-mass spectrometry (GC-MS) with headspace solid-phase microextraction (HS-SPME).

Within the context of the invention, the meat experience may be mimicked when the color of the meat substitute is similar with the color of meat. The color of meat is determined by the concentration of heme-containing protein and/or by its oxidation state in the meat. Therefore, the total amount of the isolated myoglobin added to the meat substitute will determine the color of the 5 meat substitute.

In an embodiment, the meat substitute, preferably a raw meat substitute, is expected to mimick the color of a meat, preferably of raw meat. Without being bound to this theory, the color of the meat substitute is mostly due to the addition of the isolated myoglobin. Preferably, the meat substitute of 10 the invention is able to mimick the color of meat to a higher degree than other meat substitutes (i.e. not according to the invention).

In a preferred embodiment, the color of the meat substitute, preferably of a raw meat substitute of the invention, is essentially stable for at least 1, 2, 3, 4, 5, 6, 7, 8, 9, 10, 11, 12, 13, 14, 15, 16, 17, 18, 19 or 20 days, preferably upon storage under constant light at 4° C. Preferably, the color of the meat substitute of the invention is stable for 1, 2, 3, 4, 5, 6, 7, 8, 9, 10, 11, 12, 13, 14, 15, 16, 17, 18, 19 or 20 days longer than the time period wherein the color of meat or other meat substitutes is stable, more preferably wherein a ΔE change from 0% up to 10% is defined as stable. Essentially stable for at least X days preferably means a change of ΔE of the meat substitute between 0% and 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20% between day 0 and day X, wherein ΔE is. Most preferably, ΔE of the meat substitute does not change by more than 10% upon storage for at least 1, 2, 3, 4, 5, 6, 7, 8, 9, 10, 11, 12, 13, 14, 15, 16, 17, 18, 19 or 20 days. The color may be assessed via absorbance measurements using spectrometry.

Within the context of the invention, the meat experience may be mimicked when the composition of the meat substitute is similar with the composition of meat. The composition of the meat substitute may be seen to be similar to the meat composition when the same or similar components are present and optionally when the quantities in which they are present is the same or similar with the quantities present in meat. For example a similar fat, protein and/or heme iron content may be present in a meat substitute as in meat.

### Adding Isolated Myoglobin

In the first aspect of the invention, the method for preparing a pet food comprises adding an isolated myoglobin. The isolated myoglobin may be added or applied by manufacturer of a pet food, the consumer of a pet food, or any other producer of a pet food or related product.

In an embodiment, the (total) weight concentration of heme-proteins in the pet food is at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or 300% higher than the average concentration of heme-proteins in the pet food product produced from traditional or common process which does not explicitly comprise a step of adding isolated myoglobin. In this context, the average concentration of heme-proteins in meat of a specific species of an animal is determined over several corresponding animals, wherein no exogeneous or isolated myoglobin has been added to the meat of the animals after harvesting. In this context, the resulting pet food product of this invention is edible for a pet defined herein, and the addition of myoglobin using the method of this invention does not introduce toxicity or affect the food safety of said pet food product.

In an embodiment, the (total) weight concentration of heme-proteins in the pet food is at least 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 6%, 7%, 8%, 9%, 10%, preferably lower than 10%, more preferably lower than 5%.

In an embodiment, the (total) weight concentration of myoglobins in the pet food is at least 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 6%, 7%, 8%, 9%, 10%, preferably lower than 10%, more preferably lower than 5%. It is understood that the myoglobins comprised in the pet food may originate from the addition of the isolated myoglobin, or from another source.

In an embodiment, the weight fraction of myoglobins in the heme-proteins in the pet food is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, the (total) weight concentration of heme-proteins in the pet food is at least 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 6%, 7%, 8%, 9%, 10%, and the weight fraction of myoglobins in the heme-proteins in the pet food is at least 90%.

In an embodiment, the (total) weight concentration of heme-proteins in the pet food is at least 0.05%, and the weight fraction of myoglobins in the heme-proteins in the pet food is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, the (total) weight concentration of heme-proteins in the pet food is at least 0.1%, and the weight fraction of myoglobins in the heme-proteins in the pet food is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, the (total) weight concentration of heme-proteins in the pet food is at least 0.5%, and the weight fraction of myoglobins in the heme-proteins in the pet food is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, the (total) weight concentration of heme-proteins in the pet food is at least 1.0%, and the weight fraction of myoglobins in the heme-proteins in the pet food is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, the (total) weight concentration of heme-proteins in the pet food is at least 3.0%, and the weight fraction of myoglobins in the heme-proteins in the pet food is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, at least 50%, 55%, 60%, 65%, 70%, 75%, 80%, 85%, 90%, or 95% of heme-proteins in the pet food originate from the addition of the isolated myoglobin (per weight). In a more preferred embodiment, at least 50%, 55%, 60%, 65%, 70%, 75%, 80%, 85%, 90%, or 95% of myoglobins in the pet food originate from the addition of the isolated myoglobin (per weight).

In an embodiment, at least 50%, 55%, 60%, 65%, 70%, 75%, 80%, 85%, 90%, or 95% of myoglobins in the pet food originate from the addition of the isolated myoglobin (per weight). In a more preferred embodiment, at least 50%, 55%, 60%, 65%, 70%, 75%, 80%, 85%, 90%, or 95% of myoglobins in the pet food originate from the addition of the isolated myoglobin (per weight).

In an embodiment, the isolated myoglobin is added as part of a composition, wherein the weight concentration of the isolated myoglobin in the composition at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%. In this context, weight concentration may be expressed as a mass of myoglobin per total volume or mass of the composition.

In an embodiment, the isolated myoglobin is added as part of a composition, wherein the weight fraction of the isolated myoglobin in the protein fraction comprised in the composition is at least 10%, at least 15%, at least 20%, at least 25%, at least 30%, at least 35%, at least 40%, at least 45%, at least 50%, at least 55%, at least 60%, at least 65%, at least 70%, 75%, at least 80%, at least 85%, at least 90%, at least 95%, at least 96%, at least 97%, at least 98%, at least 99%. In this context, weight fraction is a dimensionless number which may be interpreted as a mass of myoglobin per mass of protein.

In an embodiment, the isolated myoglobin is in the form of a liquid formulation (i.e. the composition may be a liquid). The amount of the isolated myoglobin needed to be added to the pet food may be calculated or estimated based on the concentration of myoglobin in the pet food before the addition and the desired final concentration of myoglobins in the pet food. In this context, the pet food before addition of the isolated myoglobin preferably contains less than 0.1%, less than 0.2%, less than 0.3%, less than 0.4%, less than 0.5%, less than 0.6%, less than 0.7%, less than 0.8%, less than 0.9%, less than 1.0% heme-proteins per weight basis.

In an embodiment, the isolated myoglobin is in the form of powder (i.e. the composition may be a powder). The amount of the isolated myoglobin needed to be added to the pet food may be calculated or estimated based on the concentration of myoglobin in the pet food before the addition and the desired final concentration of myoglobins in the pet food. In this context, the pet food before addition of the isolated myoglobin preferably contains less than 0.1%, less than 0.2%, less than 0.3%, less than 0.4%, less than 0.5%, less than 0.6%, less than 0.7%, less than 0.8%, less than 0.9%, less than 1.0% heme-proteins per weight basis. A tool, such as a container, a cup, a spoon, a blender, a mixer, may be used to add the isolated myoglobin. The isolated myoglobin may be added directly to the pet food, or may be dissolved in an editable solvent, prior to be added to the pet food.

In an embodiment, the isolated myoglobin is in the form of solid shapes, wherein the solid shaped myogloblin has a pre-determined weight and/or volume. The amount of the isolated myoglobin needed to be added to the pet food may be calculated or estimated based on the concentration of the isolated myoglobin, and/or the volume of the solid-shaped isolated myoglobin, the total weight of the pet food and the desired final amount of myoglobins in the pet food. In this context, the pet food before addition of the isolated myoglobin preferably contains less than 0.1%, less than 0.2%, less than 0.3%, less than 0.4%, less than 0.5%, less than 0.6%, less than 0.7%, less than 0.8%, less than 0.9%, less than 1.0% heme-proteins per weight basis. A tool, such as a container, a cup, a spoon, a blender, a mixer, may be used to add the isolated myoglobin. The isolated myoglobin may be added directly to the pet food, or may be dissolved in a food grade solvent, prior to be added to the pet food.

In an embodiment, the pet food is meat substitute, wherein the isolated myoglobin is added to cross-link with another substance, for example a pea vicilin protein.

### Isolated Myoglobin

In an embodiment, the method of this invention comprises adding isolated myoglobin, wherein the isolated myoglobin is a recombinant myoglobin produced from fermentation by a microorganism which has been genetically modified to express said recombinant myoglobin. Myoglobins which may be represented by SEQ ID NO: 1-6 may be encoded by nucleic acids which may be represented by SEQ ID NO: 7-12, respectively, although it is understood that these nucleic acids sequence may need to be codon-optimized in order to be expressed by the microorganism. Example 1 gives more details on the myoglobins used, whereas Example 2 provides a fermentation by a microorganism, namely by a Pichia pastoris strain.

In this application, the numbering of the myoglobin amino acid sequences originating from rat (SEQ ID NO: 1), rabbit (SEQ ID NO: 2), mouse (SEQ ID NO: 3), steppe mammoth (SEQ ID NO: 4) and woolly mammoth (SEQ ID NO:5) used herein begins with methionine or Met or M at position 1. All these amino acid sequences SEQ ID NO: 1-5 count 154 amino acid residues.

The numbering of the myoglobin amino acid sequence originating from tuna (SEQ ID NO: 6) used herein is slightly different from the consecutive numbering indicated in the sequence listing, as this sequence only counts 147 amino acid residues. In order to compare SEQ ID NO: 6 directly with SEQ ID NO: 1-5, SEQ ID NO: 6 is also attributed 154 positions, wherein eight positions are considered to be gaps (2, 3, 6, 7, 51, 120, 121).

A microorganism may be a prokaryote, a eukaryote or a filamentous fungus. A prokaryote may be a bacterium. The bacterium may be a Gram positive/Gram negative bacterium slected from the following list: Absidia, Achromobacter, Acinetobacter, Aeribacillus, Aneurinibacillus, Agrobacterium, Aeromonas, Alcaligenes, Arthrobacter, Arzoarcus, Azomonas, Azospirillum, Azotobacter, Bacillus, Beijerinckia, Bradyrhizobium, Brevibacills, Burkholderia, Byssochlamys, Citrobacter, Clostridium, Comamonas, Cupriavidus, Corynebacterium, Deinococcus, Escherichia, Enterobacter, Flavobacterium, Fusobacterium, Gossypium, Klebsiella, Lactobacillus, Listeria, Megasphaera, Micrococcus, Mycobacterium, Norcadia, Porphyromonas, Propionibacterium, Pseudomonas, Ralstonia, Rhizobium, Rhodopseudomonas, Rhodospirillum, Rodococcus, Roseburia, Shewanella, Streptomycetes, Xanthomonas, Xylella, Yersinia, Treponema, Vibrio, Streptococcus, Lactococcus, Zymomonas, Staphylococcus, Salmonella, Sphingomonas, Sphingobium, Novosphingobium, Brucella and Microscilla. Preferred bacteria include Aeribacillus pallidus, Aneurinibacillus terranovensis, Bacillus subtilis, Bacillus amyloliquefaciens, Bacillus coagulans, Bacillus licheniformis, Bacillus megaterium, Bacillus halodurans, Bacillus pumilus, Brevibacillus thermoruber, Brevibacillus panacihumi, Cupriavidus basilensis, G. Iraustophilus, Gluconobacter oxydans, Caulobacter crescentus CB 15, Methylobacterium extorquens, Rhodobacter sphaeroides, Pelotomaculum thermopropionicum, Pseudomonas zeaxanthinifaciens, Pseudomonas putida, Paracoccus denitrificans, Escherichia coli, Corynebacterium glutamicum, Staphylococcus carnosus, Streptomyces lividans, Sinorhizobium melioti, Sphingobium sp., Novosphingobium sp., Sphingomonas henshuiensis, and Rhizobium radiobacter. A preferred bacterium is Escherichia coli. Preferred Escherichia coli strains include: 58, 679, WG1, DH5a, TG1, TOP10, K12, BL21, BL21 DE3, XL1-Blue, XL10-Gold, TB1, REG-12, W945, HB101, DH1, DP50, AB284, JC9387, AG1, C600, Cavalli Hfr, Y10.

A eukaryote may be a yeast or a filamentous fungus. Preferred yeasts include Saccharomyces, Kluyveromyces, Candida, Pichia, Schizosaccharomyces, Hansenula, Kloeckera, Schwanniomyces, Yarrowia, Cryptococcus, Debaromyces, Saccharomycesopsis, Saccharomycodes, Wickerhamia, Debayomyces, Hanseniaspora, Ogataea, Kuraishia, Komagataella, Metschnikowia, Williopsis, Nakazawaea, Torulaspora, Bullera, Rhodotorula, Sporobolomyces. Within yeasts, the species Kluyveromyces lactis, Saccharomyces cerevisiae, Hansenula polymorpha (also known as Ogataea henricii), Yarrowia lipolytica, Candida tropicalis and Pichia pastoris (also known as Komagataella phaffii) are preferred. Preferred Pichia strains are selected from the following list: Bg09, Bg10, Bg11, Bg12 (exemplified), Bg20, Bg21, Bg22, Bg23, Bg24, Bg25, Bg26, Bg40, Bg43, Bg44, Bg45, Y-11430, X-33, GS115, KM71, SMD1168, SMD1165, MC100-3, most preferred Bg10 and derivatives. Preferred Saccharomyces strains are selected from the following list: S288C, CEN.PK family, CBS 2354, ATCC 2360, ATCC 4098, ATCC 4124, ATCC 4126, ATCC 4127, ATCC 4921, ATCC 7754, ATCC 9763, ATCC 20598, ATCC 24855, ATCC 24858, ATCC 24860, ATCC 26422, ATCC 46523, ATCC 56069, ATCC 60222, ATCC 60223, ATCC 60493, ATCC 66348, ATCC 66349, ATCC 96581. A preferred yeast is a Pichia strain, more preferably Pichia pastoris.

A filamentous fungus may be selected from the following list including: Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallinastix, Neurospora, Paecilomyces, Penicillium, Piromyces, Panerochaete, Pleurotus, Schizophyllum, Talaromyces, Thermoascus, Thielavia, Tolypocladium, Ustilago and Trichoderma. Preferred filamentous fungus are selected from the following list: Aspergillus niger, Aspergillus nidulans, Aspergillus fumigatus, Aspergillus oryzae, Aspergillus vadensis, Penicillium chrysogenum, Penicillium citrinum, Penicillium rubens, Penicillium oxalicum, Penicillium subrubescens, Rasamsonia emersonii, Talaromyces emersonii, Acremonium chrysogenum, Trichoderma reesei, Aspergillus sojae, and Chrysosporium lucknowense. Preferred strains of filamentous fungus are selected from the following list: Aspergillus niger CBS 513.88, N593, CBS 120.49, N402, ATCC 1015 Aspergillus oryzae ATCC 20423, IFO 4177, ATCC 1011, ATCC 9576, ATCC 14488-14491, ATCC 11601, ATCC12892, Aspergillus vadensis CBS 113365, CBS 102787, IMI 142717, IBT 24658, CBS 113226, Penicillium chrysogenum CBS 455.95, Penicillium citrinum ATCC 38065, Penicillium chrysogenum P2, Wisconsin 54-1255, Penicillium subrubescens CBS 132785, FBCC 1632, Talaromyces emersonii CBS 393.64, Acremonium chrysogenum ATCC 36225 or ATCC 48272, Trichoderma reesei ATCC 26921 or ATCC56765 or ATCC 26921, Aspergillus sojae ATCC11906, Chrysosporium lucknowense ATCC44006. In a preferred embodiment, Aspergillus is used as a filamentous fungus. More preferably, an Aspergillus niger strain is used.

In an embodiment, the microorganism used in the fermentation may be a bacterium, a yeast, a filamentous fungus or a cultured mammalian cell line, preferably Escherichia coli or Saccharomyces cerevisiae. In this context, single, isolated, cultured mammalian cells may be considered as microorganisms. In a further embodiment, the microorganism may be a bacterium, a yeast or a filamentous fungus. The microorganisms in the context of this invention are useful for the production of a myoglobin. Accordingly, in a further aspect, the invention provides a method for the production of a myoglobin as defined herein, comprising culturing the microorganisms in a suitable medium and optionally recovering the microorganism and/or myoglobin. Optionally, the produced myoglobin does not comprise a signal peptide as defined elsewhere herein.

Cell culturing may be performed for a duration of 14, 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5 or 1 days, wherein said duration may deviate by 20%. Preferably, cell culturing is performed for a duration of 14, 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5 or 1 days, wherein said duration may deviate by 10%. More preferably, the cell culturing is performed for 5 days, wherein said duration may deviate by 20%, most preferably by 10%.

The cell culturing will typically result in a production of at least 100 mg/L, 200 mg/L, 300 mg/L, 400 mg/L, 500 mg/L, 600 mg/L, 700 mg/L, 800 mg/L, 900 mg/L, 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 50 g/L, 75 g/L, 100 g/L, 200 g/L or 300 g/L of a myoglobin.

The cell culturing will typically result in at least 10%, 20%, 25%, 30%, 35%, 40%, 45%, 50%, 55%, 60%, 65%, 70%, 75%, 80%, 85%, 90%, 95%, or 100%, preferably at least 20%, more preferably at least 40%, most preferably at least 60% of the carbon source in the growth medium being converted to a myoglobin.

Cell culturing may also be performed by implementation of a multiple step, preferably a two-step, culture method. For example, a production step of a myoglobin may be preceded by a cellular biomass growth step, wherein only limited production or no production is taking place. The different steps may be carried out using different culture modes and/or different growth media and/or different culture process parameter values, depending on the goal of each step and/or the cultured cell. The biomass during the production step may or may not be actively growing.

The host cells and/or myoglobin may optionally be recovered from the culture medium. When present intracellularly, the myoglobin may optionally be recovered from the recovered cellular biomass. Optionally, the recovered myoglobin is purified. Preferably, purification of the myoglobin will result in a purity of at least 70%, more preferably at least 75%, 80%, 85%, 90%, 95%, 96%, 97%, 98%, 99%, most preferably in a myoglobin that is substantially pure.

In an embodiment, the microorganism produces the myoglobin extracellularly. The myoglobin is transported out of the host cell after it is synthesized in the host cell. In this context, both secretory and extracellular fermentations are considered to be extracellular productions. Without being bound to this theory, an extracellular production process has the advantage that the downstream processing to recover the produced myoglobin is more convenient, efficient and/or effective.

Furthermore, an extracellular production process may result in a composition comprising the myoglobin with a high purity such as at least 60%, 65%, 70%, 75%, 80%, 85%, 90%, 95%, 96%, 97%, 98%, or 99% with minimal downstream processing compared to intracellular methods wherein the myoglobin is not transported out of the host cell after its synthesis. Purity may be measured as the weight percentage of the total protein fraction in the cell-free supernatant obtained at the end of a process or extracellular process according to the invention. Without being bound to this theory, an extracellular production process has the advantage that the optional step of recovering myoglobin does not comprise lysing the host cell. As a result, the extracellular process may result in a composition having a low concentration of nucleic acids originating from the host cell.

The relevant downstream processing technology that may be suitable for recovery and/or purification will depend on whether the myoglobin is accumulated within the cultured cells or excreted. Said processing technology and the associated choice will be known to the skilled person and is discussed, for example, in Wesselingh, J. A and Krijgsman, J., 1st edition, Downstream Processing in Biotechnology, Delft Academic Press, N L, 2013. In a recovery process, the biomass may be recovered from the culture medium using e.g. centrifugation or filtration. If the produced myoglobin is accumulated within the cells, it can then be recovered and/or purified from the biomass. If it is excreted, it can be recovered from the cell-free medium or, if the biomass separation step is skipped, directly from the culture broth. Recovery and/or purification may be performed according to any conventional recovery or purification methodology known in the art. Methods for recovery and/or purification of proteins are known to the skilled person and are discussed in standard handbooks, such as Sambrook and Russel, Molecular Cloning: A Laboratory Manual, 3rd edition, Cold Spring Harbor Laboratory, Cold Spring Harbor Laboratory Press, NY, 2001 or Ausubel F. et al, eds., Current protocols in molecular biology, Green Publishing and Wiley Interscience, NY, 2003. Examples of widely used recovery and/or purification methods include chromatographic methods such as gel-filtration chromatography, ion-exchange chromatography, immunoaffinity chromatography, metal affinity chromatography, gel-filtration chromatography, fractionation with precipitants such as ammonium sulfate and polyethylene glycol, gel electrophoresis and salting out and dialysis. Preferably, metal affinity chromatography or size-exclusion chromatography is used. Recovery and/or purification may optionally be enhanced by linking the enzyme polypeptide to a sequence that facilitates purification, such as with a GST domain, using well-known molecular toolbox techniques. Optionally, the sequence that facilitates purification and/or signal peptide that facilitates excretion of the myoglobin is removed from the final product using techniques known in the art, for example proteolysis by endopeptidases targeting a linker between the sequence that facilitates purification and/or the signal peptide and the myoglobin. In some embodiments, the enzyme polypeptide is linked (fused) to a hexa-histidine peptide, such as the tag provided in a pET23a (+) vector (Genescript Biotech, Leiden, The Netherlands), among others, many of which are commercially available. As, for example, described in Gentz et al., Proc. Natl. Acad. Sci. USA 86:821-824 (1989), hexa-histidine peptide provides for convenient purificaton of the fusion protein.

In a preferred embodiment, the isolated myoglobin is obtained via recovering and/or purifing from the culture medium. This may be realized continuously with the production process or subsequently to it. In a preferred embodiment, the isolated myoglobin is obtained via recovering and/or purifing from the cultured cells. A filter may be used for the purification of the recovered myoglobin. This may be realized continuously with the production process, by harvesting fractions of growing cells, or subsequently to it.

In an embodiment, the isolated myoglobin is sterilized, shredded, spray dryed, freeze dryed, blended, shaped, cubed, dosed or packed. Sterilization refers to any process that removes, kills, or deactivates all forms of life (particularly microorganisms such as fungi, bacteria, spores, and unicellular eukaryotic organisms) and other biological agents such as prions present in or on a specific surface, object, or fluid. Sterilization can be achieved through various means, including heat, chemicals, irradiation, high pressure, and filtration. Freeze drying, also known as lyophilization or cryodesiccation, is a low temperature dehydration process that involves freezing the product, lowering pressure, then removing the ice by sublimation. Packing aims to provide a protection for the product, to tamper resistance and to provide physical, chemical or biological needs. Packing may also contain nutrition facts, characteristics of the products, and an instruction/guide for use of the product.

In a preferred embodiment, the cultured host cells used in fermentation are immobilized. Immobilization of cells may be achieved by any means known to the skilled person as discussed in standard handbooks such as Guisan, J. M., Bolivar, J. M., López-Gallego, F., Rocha-Martin, J. (Eds.), Immobilization of Enzymes and Cells: Methods and Protocols, Springer US, USA, 2020. Typically, the host cells can be immobilized to a semi-solid or solid support by three different methods. The first method involves polymerizing or solidifying a spore- or cell-containing solution. Examples of polymerizable or solidifiable solutions include alginate, A-carrageenan, chitosan, polyacrylamide, polyacrylamide-hydrazide, agarose, polypropylene, polyethylene glycol, dimethyl acrylate, polystyrene divinyle benzene, polyvinyl benzene, polyvinyl alcohol, epoxy carrier, cellulose, cellulose acetate, photocrosslinkable resin, prepolymers, urethane, and gelatin. The second method involves cell adsorption onto a support. Examples of such supports include bone char, cork, clay, resin, sand porous alumina beads, porous brick, porous silica, celite, or wood chips. The host cells can colonize the support and form a biofilm. The third method involves the covalent coupling of the host cells to a support using chemical agents like glutaraldehyde, o-dianisidine (U.S. Pat. No. 3,983,000), polymeric isocyanates (U.S. Pat. No. 4,071,409), silanes (U.S. Pat. Nos. 3,519,538 and 3,652,761), hydroxyethyl acrylate, transition metal-activated supports, cyanuric chloride, sodium periodate, toluene, and the like. Cultured host cells can be immobilized in any phase of their growth, for example after a desired cell density in the culture has been reached. Suitable culture modes and/or different culture process parameter values will be known to the skilled person and are discussed in standard handbooks, such as Colin R. Phillips C.R., Poon Y. C., Immobilization of Cells: In Biotechnology Monographs book series (Biotechnology, volume 5), Springer, Berlin, Germany, 1988; Tampion J., Tampion M. D., Immobilized Cells: Principles and Applications, Cambridge University Press, U K, 1987. Preferably, immobilized cells are cultured in packed bed bioreactors, also known as plug-flow bioreactors, or expanded (fluidized) bed bioreactors. Suitable growth media and recovery and/or purification methods are further discussed elsewhere herein.

In an embodiment, an isolated myoglobin is a myoglobin which is not comprised in a cell or a tissue before the addition. In other words, the isolated myoglobin is added as a “separate” (or isolated) protein which is not comprised in a cell or a tissue. Thus, isolated in this context does not refer to the origin of the myoglobin (i.e. isolated from a source such as cell or a tissue), but to the fact that a separate protein is added. It is of course understood that the isolated myoglobin may become part of a cell or a tissue once it has been added to the pet food.

An isolated myoglobin may be derived from rat, rabbit, mouse, mammoth or tuna. Preferably, the amino acids sequence is derived from rat, rabbit, mouse, mammoth or tuna, by addition, deletion and/or substitution of at least one amino acid. Addition, deletion and/or substitutions of two, three, four, five, six, seven, eight, nine or ten amino acids is also contemplated by the invention. Examples of myoglobin amino acid sequence from rat, rabbit, mouse, mammoth or tuna have been disclosed later on by a given SEQ ID NO:1-6. Such isolated myoglobin derived from rat, rabbit, mouse, mammoth or tuna may also exert at least a detectable level of an activity of an isolated myoglobin as explained later herein. A mammoth may be any species in the genus Mammuthus, such as a woolly mammoth (Mammuthus primigenius) or a steppe mammoth (Mammuthus trogontherii), in the context of this application, unless explicitly stated otherwise.

In an embodiment, the isolated myoglobin is a rat myoglobin, a rabbit myoglobin, a mouse myoglobin, a mammoth myoglobin or a tuna myoglobin, or a myoglobin derived from any of these myoglobins. In an embodiment, the isolated myoglobin is rat myoglobin or derived therefrom. In an embodiment, the isolated myoglobin is rabbit myoglobin or derived therefrom. In an embodiment, the isolated myoglobin is mouse myoglobin or derived therefrom. In an embodiment, the isolated myoglobin is steppe mammoth myoglobin or derived therefrom. In an embodiment, the isolated myoglobin is woolly mammoth myoglobin or derived therefrom. In an embodiment, the the isolated myoglobin is tuna myoglobin or derived therefrom.

In an embodiment, the isolated myoglobin may be represented by any one of SEQ ID NO: 1, 2, 3, 4, 5 or 6, preferably by SEQ ID NO: 1 or 2.

In an embodiment, the isolated myoglobin is from a rat as defined earlier herein, preferably a myoglobin having at least 90% sequence identity with SEQ ID NO: 1, more preferably a myoglobin having at least 90% sequence identity with SEQ ID NO: 1, in combination with G at position 6, Q at position 9, M at position 10, N at position 13, I at position 14, L at position 22, A at position 23, Q at position 27, L at position 30, I at position 31, G at position 32, K at position 35, A at position 36, E at position 42, K at position 43, N at position 49, S at position 52, E at position 53, E at position 54, E at position 55, S at position 58, H at position 65, C at position 67, A at position 72, T at position 75, K at position 79, Q at positon 82, Q at position 92, S at position 93, T at position 96, V at position 102, E at position 110, V at position 111, Q at position 114, L at position 116, K at position 117, K at position 118, R at position 119, S at position 121, G at position 122, D at position 123, A at position 128, G at position 130, S at position 133, I at position 143, A at position 145, K at position 146, E at position 149, L at position 150, or Q at position 153.

In an embodiment, the isolated myoglobin is from a rabbit as defined earlier herein, preferably a myoglobin having at least 90% sequence identity with SEQ ID NO: 2, more preferably a myoglobin having at least 90% sequence identity with SEQ ID NO: 2, in combination with A at position 6, Q at position 9, L at position 10, N at position 13, V at position 14, L at position 22, A at position 23, Q at position 27, L at position 30, I at position 31, G at position 32, H at position 35, T at position 36, E at position 42, K at position 43, H at position 49, S at position 52, E at position 53, D at position 54, E at position 55, A at position 58, H at position 65, N at position 67, A at position 72, A at position 75, K at position 79, H at positon 82, Q at position 92, S at position 93, T at position 96, V at position 102, E at position 110, A at position 111, H at position 114, L at position 116, H at position 117, S at position 118, R at position 119, P at position 121, G at position 122, D at position 123, A at position 128, A at position 130, S at position 133, I at position 143, A at position 145, K at position 146, E at position 149, L at position 150, or Q at position 153.

In an embodiment, the isolated myoglobin is from a mouse as defined earlier herein, preferably a myoglobin having at least 90% sequence identity with SEQ ID NO: 3, more preferably a myoglobin having at least 90% sequence identity with SEQ ID NO: 3, in combination with G at position 6, Q at position 9, L at position 10, N at position 13, V at position 14, L at position 22, A at position 23, Q at position 27, L at position 30, I at position 31, G at position 32, K at position 35, T at position 36, D at position 42, K at position 43, N at position 49, S at position 52, E at position 53, E at position 54, D at position 55, G at position 58, H at position 65, C at position 67, A at position 72, T at position 75, K at position 79, Q at positon 82, Q at position 92, S at position 93, T at position 96, V at position 102, E at position 110, I at position 111, E at position 114, L at position 116, K at position 117, K at position 118, R at position 119, S at position 121, G at position 122, D at position 123, A at position 128, G at position 130, S at position 133, I at position 143, A at position 145, K at position 146, E at position 149, L at position 150, or Q at position 153.

In an embodiment, the isolated myoglobin is from a steppe mammoth as defined earlier herein, preferably a myoglobin having at least 90% sequence identity with SEQ ID NO: 4, more preferably a myoglobin having at least 90% sequence identity with SEQ ID NO: 4 in combination with G at position 6, E at position 9, L at position 10, K at position 13, T at position 14, I at position 22, P at position 23, L at position 27, F at position 30, V at position 31, T at position 35, G at position 36, E at position 42, K at position 43, H at position 49, T at position 52, E at position 53, G at position 54, E at position 55, A at position 58, Q at position 65, V at position 67, A at position 72, G at position 75, K at position 79, H at position 82, Q at position 84, A at position 85, I at position 87, Q at position 88, P at position 89, H at position 92, S at position 93, T at position 96, I at position 102, D at position 110, A at position 111, H at position 114, L at position 116, Q at position 117, S at position 118, P at position 121, A at position 122, E at position 123, A at position 128, G at position 130, K at position 133, I at position 143, A at position 145, K at position 146, E at position 149, L at position 150, or Q at position 153.

In an embodiment, the isolated myoglobin is from a woolly mammoth as defined earlier herein, preferably a myoglobin having at least 90% sequence identity with SEQ ID NO: 5, more preferably a myoglobin having at least 90% sequence identity with SEQ ID NO: 5 in combination with G at position 6, E at position 9, L at position 10, K at position 13, T at position 14, I at position 22, P at position 23, L at position 27, F at position 30, V at position 31, T at position 35, G at position 36, E at position 42, K at position 43, H at position 49, T at position 52, E at position 53, G at position 54, E at position 55, A at position 58, Q at position 65, V at position 67, A at position 72, G at position 75, K at position 79, H at position 82, Q at position 84, A at position 85, I at position 87, Q at position 88, P at position 89, Q at position 92, S at position 93, T at position 96, I at position 102, D at position 110, A at position 111, H at position 114, L at position 116, Q at position 117, S at position 118, P at position 121, A at position 122, E at position 123, A at position 128, G at position 130, K at position 133, I at position 143, A at position 145, K at position 146, E at position 149, L at position 150, or Q at position 153.

In an embodiment, the isolated myoglobin is from a tuna as defined earlier herein, preferably a myoglobin having at least 90% sequence identity with SEQ ID NO: 6, more preferably a myoglobin having at least 90% sequence identity with SEQ ID NO: 6 in combination with D at position 9, A at position 10, K at position 13, C at position 14, Y at position 22, T at position 23, G at position 27, L at position 30, T at position 31, K at position 35, E at position 36, K at position 42, L at position 43, G at position 49, A at position 52, Q at position 53, A at position 54, D at position 55, G at position 58, H at position 65, A at position 67, K at position 72, E at position 75, A at position 79, S at position 82, A at position 84, A at position 85, L at position 87, K at position 88, P at position 89, N at position 92, S at position 93, T at position 96, I at position 102, E at position 110, V at position 111, K at position 114, M at position 116, H at position 117, E at position 118, A at position 122, G at position 123, Q at position 128, T at position 130, R at position 133, L at position 143, A at position 145, N at position 146, E at position 149, L at position 150, or S at position 153.

In an embodiment, the isolated myoglobin may be represented by one of the following amino acid sequences:
a) having at least 90% sequence identity with SEQ ID NO: 1, and having at least one of the following amino acid combinations:
   M at position 10, and/or
   A at position 36, and/or
   S at position 58, and/or
   M at position 10 and A at position 36, and/or
   M at position 10 and S at position 58, and/or
   A at position 36 and S at position 58, and/or
   M at position 10 and A at position 36 and S at position 58;
b) having at least 90% sequence identity with SEQ ID NO: 2, and having an amino acid A at position 6;
c) having at least 90% sequence identity with SEQ ID NO: 3, and having at least one of the following amino acid combinations:
   D at position 55, and/or
   I at position 111, and/or
   E at position 114, and/or
   D at position 55 and I at position 111, and/or
   D at position 55 and E at position 114, and/or
   I at position 111 and E at position 114, and/or
   D at position 55 and I at position 111 and E at position 114;
d) having at least 90% identity with SEQ ID NO: 4 or 5, and having F at position 30 and/or Q at position 65;
e) having at least 90% identity with SEQ ID NO: 6, and having H at position 65 and/or position 94;
f) having at least 90% identity with SEQ ID NO: 1, 2 or 3, and having at least one of the following amino acid combinations;
   L at position 22, and/or
   A or T at position 36, and/or
   G at position 122;
   L at position 22 and A or T at position 36, and/or
   L at position 22 and G at position 122; and/or
   A or T at position 36 and G at position 122, and/or
   L at position 22 and A or T at position 36 and G at position 122;
g) having at least 90% identity with any one from SEQ ID NO: 1 to 5, and having I at position 87;
h) having at least 90% identity with SEQ ID NO: 1 or 3, and having at least one or more of the following amino acids or any combinations thereof;
   N at position 49, and/or
   E at position 54, and/or
   C at position 67, and/o
   T at position 75, and/or
   Q at position 82, and/or
   K at position 117, and/or
   K at position 118, and/or
   S at position 121,
i) having at least 90% identity with SEQ ID NO: 4, and having at least one of the following amino acid combinations:
   H at position 92, and/or
   H at position 92 and F at position 30, and/or
   H at position 92 and Q at position 65, and/or;
   H at position 92 and F at position 30 and Q at position 65.

In an embodiment, the isolated myoglobin is a recombinant myoglobin, wherein the recombinant myoglobin is from a rat as defined earlier herein, preferably a myoglobin having at least 90% sequence identity with SEQ ID NO: 1, more preferably a myoglobin having at least 90% sequence identity with SEQ ID NO: 1, in combination with G at position 6, Q at position 9, M at position 10, N at position 13, I at position 14, L at position 22, A at position 23, Q at position 27, L at position 30, I at position 31, G at position 32, K at position 35, A at position 36, E at position 42, K at position 43, N at position 49, S at position 52, E at position 53, E at position 54, E at position 55, S at position 58, H at position 65, C at position 67, A at position 72, T at position 75, K at position 79, Q at positon 82, Q at position 92, S at position 93, T at position 96, V at position 102, E at position 110, V at position 111, Q at position 114, L at position 116, K at position 117, K at position 118, R at position 119, S at position 121, G at position 122, D at position 123, A at position 128, G at position 130, S at position 133, I at position 143, A at position 145, K at position 146, E at position 149, L at position 150, or Q at position 153.

In an embodiment, the isolated myoglobin is a recombinant myoglobin, wherein the recombinant myoglobin is from a rabbit as defined earlier herein, preferably a myoglobin having at least 90% sequence identity with SEQ ID NO: 2, more preferably a myoglobin having at least 90% sequence identity with SEQ ID NO: 2, in combination with A at position 6, Q at position 9, L at position 10, N at position 13, V at position 14, L at position 22, A at position 23, Q at position 27, L at position 30, I at position 31, G at position 32, H at position 35, T at position 36, E at position 42, K at position 43, H at position 49, S at position 52, E at position 53, D at position 54, E at position 55, A at position 58, H at position 65, N at position 67, A at position 72, A at position 75, K at position 79, H at positon 82, Q at position 92, S at position 93, T at position 96, V at position 102, E at position 110, A at position 111, H at position 114, L at position 116, H at position 117, S at position 118, R at position 119, P at position 121, G at position 122, D at position 123, A at position 128, A at position 130, S at position 133, I at position 143, A at position 145, K at position 146, E at position 149, L at position 150, or Q at position 153.

In an embodiment, the isolated myoglobin is a recombinant myoglobin, wherein the recombinant myoglobin is from a mouse as defined earlier herein, preferably a myoglobin having at least 90% sequence identity with SEQ ID NO: 3, more preferably a myoglobin having at least 90% sequence identity with SEQ ID NO: 3, in combination with G at position 6, Q at position 9, L at position 10, N at position 13, V at position 14, L at position 22, A at position 23, Q at position 27, L at position 30, I at position 31, G at position 32, K at position 35, T at position 36, D at position 42, K at position 43, N at position 49, S at position 52, E at position 53, E at position 54, D at position 55, G at position 58, H at position 65, C at position 67, A at position 72, T at position 75, K at position 79, Q at positon 82, Q at position 92, S at position 93, T at position 96, V at position 102, E at position 110, I at position 111, E at position 114, L at position 116, K at position 117, K at position 118, R at position 119, S at position 121, G at position 122, D at position 123, A at position 128, G at position 130, S at position 133, I at position 143, A at position 145, K at position 146, E at position 149, L at position 150, or Q at position 153.

In an embodiment, the isolated myoglobin is a recombinant myoglobin, wherein the recombinant myoglobin is from a steppe mammoth as defined earlier herein, preferably a myoglobin having at least 90% sequence identity with SEQ ID NO: 4, more preferably a myoglobin having at least 90% sequence identity with SEQ ID NO: 4 in combination with G at position 6, E at position 9, L at position 10, K at position 13, T at position 14, I at position 22, P at position 23, L at position 27, F at position 30, V at position 31, T at position 35, G at position 36, E at position 42, K at position 43, H at position 49, T at position 52, E at position 53, G at position 54, E at position 55, A at position 58, Q at position 65, V at position 67, A at position 72, G at position 75, K at position 79, H at position 82, Q at position 84, A at position 85, I at position 87, Q at position 88, P at position 89, H at position 92, S at position 93, T at position 96, I at position 102, D at position 110, A at position 111, H at position 114, L at position 116, Q at position 117, S at position 118, P at position 121, A at position 122, E at position 123, A at position 128, G at position 130, K at position 133, I at position 143, A at position 145, K at position 146, E at position 149, L at position 150, or Q at position 153.

In an embodiment, the isolated myoglobin is a recombinant myoglobin, wherein the recombinant myoglobin is from a woolly mammoth as defined earlier herein, preferably a myoglobin having at least 90% sequence identity with SEQ ID NO: 5, more preferably a myoglobin having at least 90% sequence identity with SEQ ID NO: 5 in combination with G at position 6, E at position 9, L at position 10, K at position 13, T at position 14, I at position 22, P at position 23, L at position 27, F at position 30, V at position 31, T at position 35, G at position 36, E at position 42, K at position 43, H at position 49, T at position 52, E at position 53, G at position 54, E at position 55, A at position 58, Q at position 65, V at position 67, A at position 72, G at position 75, K at position 79, H at position 82, Q at position 84, A at position 85, I at position 87, Q at position 88, P at position 89, Q at position 92, S at position 93, T at position 96, I at position 102, D at position 110, A at position 111, H at position 114, L at position 116, Q at position 117, S at position 118, P at position 121, A at position 122, E at position 123, A at position 128, G at position 130, K at position 133, I at position 143, A at position 145, K at position 146, E at position 149, L at position 150, or Q at position 153.

In an embodiment, the isolated myoglobin is a recombinant myoglobin, wherein the recombinant myoglobin is from a tuna as defined earlier herein, preferably a myoglobin having at least 90% sequence identity with SEQ ID NO: 6, more preferably a myoglobin having at least 90% sequence identity with SEQ ID NO: 6 in combination with D at position 9, A at position 10, K at position 13, C at position 14, Y at position 22, T at position 23, G at position 27, L at position 30, T at position 31, K at position 35, E at position 36, K at position 42, L at position 43, G at position 49, A at position 52, Q at position 53, A at position 54, D at position 55, G at position 58, H at position 65, A at position 67, K at position 72, E at position 75, A at position 79, S at position 82, A at position 84, A at position 85, L at position 87, K at position 88, P at position 89, N at position 92, S at position 93, T at position 96, I at position 102, E at position 110, V at position 111, K at position 114, M at position 116, H at position 117, E at position 118, A at position 122, G at position 123, Q at position 128, T at position 130, R at position 133, L at position 143, A at position 145, N at position 146, E at position 149, L at position 150, or S at position 153.

In an embodiment, the isolated myoglobin is a recombinant myoglobin, wherein the recombinant myoglobin may be represented by one of the following amino acid sequences:
a) having at least 90% sequence identity with SEQ ID NO: 1, and having at least one of the following amino acid combinations:
   M at position 10, and/or
   A at position 36, and/or
   S at position 58, and/or
   M at position 10 and A at position 36, and/or
   M at position 10 and S at position 58, and/or
   A at position 36 and S at position 58, and/or
   M at position 10 and A at position 36 and S at position 58;
b) having at least 90% sequence identity with SEQ ID NO: 2, and having an amino acid A at position 6;
c) having at least 90% sequence identity with SEQ ID NO: 3, and having at least one of the following amino acid combinations:
   D at position 55, and/or
   I at position 111, and/or
   E at position 114, and/or
   D at position 55 and I at position 111, and/or
   D at position 55 and E at position 114, and/or
   I at position 111 and E at position 114, and/or
   D at position 55 and I at position 111 and E at position 114;
d) having at least 90% identity with SEQ ID NO: 4 or 5, and having F at position 30 and/or Q at position 65;
e) having at least 90% identity with SEQ ID NO: 6, and having H at position 65 and/or position 94;
f) having at least 90% identity with SEQ ID NO: 1, 2 or 3, and having at least one of the following amino acid combinations;
   L at position 22, and/or
   A or T at position 36, and/or
   G at position 122;
   L at position 22 and A or T at position 36, and/or
   L at position 22 and G at position 122; and/or
   A or T at position 36 and G at position 122, and/or
   L at position 22 and A or T at position 36 and G at position 122;
g) having at least 90% identity with any one from SEQ ID NO: 1 to 5, and having I at position 87;
h) having at least 90% identity with SEQ ID NO: 1 or 3, and having at least one or more of the following amino acids or any combinations thereof;
   N at position 49, and/or
   E at position 54, and/or
   C at position 67, and/or
   T at position 75, and/or
   Q at position 82, and/or
   K at position 117, and/or
   K at position 118, and/or
   S at position 121.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 1 in combination with at least one of the following amino acid combinations:
M at position 10, and/or
A at position 36, and/or
S at position 58, and/or
M at position 10 and A at position 36, and/or
M at position 10 and S at position 58, and/or
A at position 36 and S at position 58, and/or
M at position 10 and A at position 36 and S at position 58.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 2 and having an amino acid A at position 6.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 3 in combination with at least one of the following amino acid combinations:
D at position 55, and/or
I at position 111, and/or
E at position 114, and/or
D at position 55 and I at position 111, and/or
D at position 55 and E at position 114, and/or
I at position 111 and E at position 114, and/or
D at position 55 and I at position 111 and E at position 114.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 4 or 5 and having F at position 30 and/or Q at position 65.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 6 and having H at position 65 and/or position 94.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 1, 2 or 3 in combination with at least one of the following amino acid combinations:
L at position 22, and/or
A or T at position 36, and/or
G at position 122;
L at position 22 and A or T at position 36, and/or
L at position 22 and G at position 122; and/or
A or T at position 36 and G at position 122, and/or
L at position 22 and A or T at position 36 and G at position 122.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 1 to 5, and having I at position 87.

In an embodiment, the amino acid sequence of the isolated myoglobin or preferably the recombinant myoglobin identified earlier herein comprises a sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with the SEQ ID NO: 1 or 3 and having at least one or more of the following amino acids or any combinations thereof;
N at position 49, and/or
E at position 54, and/or
C at position 67, and/or
T at position 75, and/or
Q at position 82, and/or
K at position 117, and/or
K at position 118, and/or
S at position 121.

### a Pet Food

In the second aspect of this invention, there is provided a pet food obtainable by the methods described herein. The pet food according to this aspect comprises a myoglobin, wherein the isolated myoglobin is a rat myoglobin, a rabbit myoglobin, a mouse myoglobin, a mammoth myoglobin, a tuna myoglobin, or a combination of any two or three or four or five of said myoglobins.

All preferences and embodiments listed for the methods for preparing a pet food of the first aspect of the invention may be applied mutatis mutandis to the pet food of the second aspect of the invention. For example, any specification of the sequence of the isolated myoglobin added during the method may be applied to the sequence of a myoglobin comprised in the pet food of the second aspect. This is evident as the pet food of the second aspect is obtainable by the method of the first aspect.

In an embodiment, the pet food, which is preferably a meat substitute, does not comprise one or more milk proteins selected from the group consisting of β-casein, κ-casein, α-S1-casein, α-S2-casein, α-lactalbumin, β-lactoglobulin, lactoferrin and transferrin, wherein said milk protein is from a woolly mammoth. In another embodiment, the meat substitute does not comprise one or more milk proteins selected from the group consisting of β-casein, κ-casein, α-S1-casein, α-S2-casein, α-lactalbumin, β-lactoglobulin, lactoferrin and transferrin, wherein said milk protein is from a mammoth.

In an embodiment, the pet food, which is preferably a meat substitute, does not comprise one or more milk proteins selected from the group consisting of β-casein, κ-casein, α-S1-casein, α-S2-casein, α-lactalbumin, β-lactoglobulin, lactoferrin and transferrin, wherein said milk protein has not been produced by a mammal or a mammalian cell.

In a more preferred embodiment, the pet food, which is preferably a meat substitute, does not comprise a milk protein such as β-casein, κ-casein, α-S1-casein, α-S2-casein, α-lactalbumin, β-lactoglobulin, lactoferrin or transferrin, wherein said milk protein is from a woolly mammoth. In another more preferred embodiment, the meat substitute does not comprise a milk protein such as β-casein, κ-casein, α-S1-casein, α-S2-casein, α-lactalbumin, β-lactoglobulin, lactoferrin or transferrin, wherein said milk protein is from a mammoth.

In another more preferred embodiment, the pet food, which is preferably a meat substitute, does not comprise a milk protein such as β-casein, κ-casein, α-S1-casein, α-S2-casein, α-lactalbumin, β-lactoglobulin, lactoferrin or transferrin, wherein said milk protein has not been produced by a mammal or a mammalian cell.

In an embodiment, the pet food, which is preferably a meat substitute, does not comprise a κ-casein from a woolly mammoth. In a further embodiment, the meat substitute does not comprise a protein represented by a sequence having at least 90%, 91%, 92%, 93%, 94%, 95%, 96%, 97%, 98%, 99% sequence identity with SEQ ID NO: 13. In a further embodiment, the pet food, which is preferably a meat substitute, does not comprise a protein represented by SEQ ID NO: 13.

In an embodiment, the pet food, which is preferably a meat substitute, does not comprise a β-casein from a woolly mammoth. In a further embodiment, the meat substitute does not comprise a protein represented by a sequence having at least 90%, 91%, 92%, 93%, 94%, 95%, 96%, 97%, 98%, 99% sequence identity with SEQ ID NO: 14. In a further embodiment, the pet food, which is preferably a meat substitute, does not comprise a protein represented by SEQ ID NO: 14.

In an embodiment, the pet food, which is preferably a meat substitute, does not comprise a symbiotic hemoglobin which has been produced by a bacterium living in symbiosis in the root nodules of a soy plant. In an embodiment, the pet food, which is preferably a meat substitute, does not comprise a leghemoglobin. In an embodiment, the isolated myoglobin disclosed herein may be the sole source of heme-containing protein present in the meat substitute. It means the meat substitute may comprise other proteins than the isolated myoglobin as disclosed herein. Examples of proteins that may be present include soy proteins.

Within the context of the invention, a heme-containing protein as defined can refer to all proteins or protein subunits that are capable of covalently or noncovalently binding a heme moiety. Heme-containing polypeptides can transport or store oxygen. Some examples of heme-containing protein include globin, hemoglobin, leghemoglobin.

In an embodiment, the pet food, which is preferably a meat substitute, is prepared by mixing (i.e. adding) from 10% up to 40% of a protein source, from 5% up to 30% of a lipid source, and from 0.05% up to 5%, preferably from 0.1% up to 5%, of an isolated myoglobin. In a more preferred embodiment, the pet food, which is preferably a meat substitute, is prepared by mixing from 20% up to 30% of a protein source, from 10% up to 20% of a lipid source, and from 0.05% up to 3%, preferably from 0.1% up to 3% of an isolated myoglobin.

In an embodiment, the pet food, which is preferably a meat substitute, is prepared by mixing (i.e. adding) from 10% up to 40% of a protein source, from 5% up to 30% of a lipid source, from 0.1% up to 5% of NaCl, from 0.1% up to 5% of a fiber source, and from 0.05% up to 5%, preferably from 0.1 up to 5%, of an isolated myoglobin. In a more preferred embodiment, the pet food, which is preferably a meat substitute, is prepared by mixing from 20% up to 30% of a protein source, from 10% up to 20% of a lipid source, from 0.5% up to 2% of NaCl, from 0.5% up to 1.5% of a fiber source, and from 0.05 up to 3%, preferably 0.1% up to 3%, of an isolated myoglobin.

Preferably, the protein source is texturized soy protein, soy protein flour, concentrate or isolate, pea protein flour, concentrate or isolate, fava bean protein, mung bean protein, textured wheat protein, rice protein or potato protein. In this context, texturized soy protein is a defatted soy flour product, also known as texturized vegetable protein (TVP) or soy meat, as is clear to the skilled person. Preferably, the lipid source is sunflower oil, coconut oil, canola oil or cocoa butter, or any plant-derived oil. Preferably, the fiber source is methylcellulose or potato starch.

In a more preferred embodiment, the pet food, which is preferably a meat substitute, is prepared by mixing from 20% up to 30% of texturized soy protein, from 10% up to 20% of sunflower plant-derived oil, from 0.5% up to 2% of NaCl, from 0.5% up to 1.5% of methylcellulose, and from 0.05% up to 5%, preferably from 0.1% up to 5%, of an isolated myoglobin. Even more preferably, the weight percentage of the isolated myoglobin in the meat substitute according to the more preferred embodiment is from 0.05% up to 5%, 4.9%, 4.8%, 4.7%, 4.6%, 4.5%, 4.4%, 4.3%, 4.2%, 4.1%, 4%, 3.9%, 3.8%, 3.7%, 3.6%, 3.5%, 3.4%, 3.3%, 3.2%, 3.1%, 3%, 2.95%, 2.9%, 2.85%, 2.8%, 2.75%, 2.7%, 2.65%, 2.6%, 2.55%, 2.5%, 2.45%, 2.4%, 2.35%, 2.3%, 2.25%, 2.2%, 2.15%, 2.1%, 2.05%, 2%, 1.95%, 1.9%, 1.85%, 1.8%, 1.75%, 1.7%, 1.65%, 1.6%, 1.55%, 1.5%, 1.45%, 1.4%, 1.35%, 1.3%, 1.25%, 1.2%, 1.15%, 1.1%, 1.05%, 1%, 0.95%, 0.9%, 0.85%, 0.8%, 0.75%, 0.7%, 0.65%, 0.6%, 0.55%, or 0.5%. Most preferably, the weight percentage of the isolated myoglobin in the meat substitute according to the more preferred embodiment is from 0.1% up to 5%, 4.9%, 4.8%, 4.7%, 4.6%, 4.5%, 4.4%, 4.3%, 4.2%, 4.1%, 4%, 3.9%, 3.8%, 3.7%, 3.6%, 3.5%, 3.4%, 3.3%, 3.2%, 3.1%, 3%, 2.95%, 2.9%, 2.85%, 2.8%, 2.75%, 2.7%, 2.65%, 2.6%, 2.55%, 2.5%, 2.45%, 2.4%, 2.35%, 2.3%, 2.25%, 2.2%, 2.15%, 2.1%, 2.05%, 2%, 1.95%, 1.9%, 1.85%, 1.8%, 1.75%, 1.7%, 1.65%, 1.6%, 1.55%, 1.5%, 1.45%, 1.4%, 1.35%, 1.3%, 1.25%, 1.2%, 1.15%, 1.1%, 1.05%, 1%, 0.95%, 0.9%, 0.85%, 0.8%, 0.75%, 0.7%, 0.65%, 0.6%, 0.55%, or 0.5%.

In an embodiment, the pet food, which is preferably a meat substitute, is prepared by mixing from 20% up to 30% texturized soy protein, from 10% up to 20% sunflower oil, from 1% up to 2% NaCl, from 0.5% up to 1.5% methylcellulose, from 50% up to 70% water and from 0.05% up to 1%, preferably from 0.2% up to 0.8%, isolated myoglobin. In a preferred embodiment, the pet food, which is preferably a meat substitute, is prepared by mixing from 23% up to 27% texturized soy protein, from 13% up to 17% sunflower oil, from 1.25% up to 1.75% NaCl, from 0.75% up to 1.25% methylcellulose, from 55% up to 60% water and from 0.45% up to 0.55% isolated myoglobin.

In an embodiment, the pet food, which is preferably a meat substitute, is prepared by mixing 25% texturized soy protein, 15% sunflower oil, 1.5% NaCl, 1.0% methylcellulose, 57% water and 0.5% isolated myoglobin, wherein these weight percentages are rounded to two significant digits.

In an embodiment, the pet food, which is preferably a meat substitute, is prepared by mixing from 15% up to 35% texturized soy protein, from 10% up to 20% sunflower oil, from 0.5% up to 2.5% NaCl, from 0.5% up to 1.5% methylcellulose, from 50% up to 70% water and from 0.5% up to 1.5% isolated myoglobin. In a preferred embodiment, the pet food, which is preferably a meat substitute, is prepared by mixing from 22% up to 27% texturized soy protein, from 13% up to 17% sunflower oil, from 1.25% up to 1.75% NaCl, from 0.75% up to 1.25% methylcellulose, from 50% up to 60% water and from 0.75% up to 1.25% isolated myoglobin.

In an embodiment, the pet food, which is preferably a meat substitute, is prepared by mixing 25% texturized soy protein, 15% sunflower oil, 1.5% NaCl, 1.0% methylcellulose, 57% water and 1.0% isolated myoglobin, wherein these weight percentages are rounded to two significant digits.

In the meat substitutes above, % refers to a weight percentage. Insofar as the weight percentages describing the composition of a meat substitute do not add up to 100%, the addition of water to a corresponding percentage is assumed.

In an embodiment, the pet food, which is preferably a meat substitute, comprises a similar amount of bioavailable iron or heme iron as the counterpart genuine meat. In an embodiment such amount is ranged from 0.3 to 20 mg bioavailable iron or heme iron per 100 gram pet food. In an embodiment such amount is ranged from 0.1 mg up to 16.5 mg, 16.17 mg, 15.84 mg, 15.51 mg, 15.18 mg, 14.85 mg, 14.52 mg, 14.19 mg, 13.86 mg, 13.53 mg, 13.2 mg, 12.87 mg, 12.54 mg, 12.21 mg, 11.88 mg, 11.55 mg, 11.22 mg, 10.89 mg, 10.56 mg, 10.23 mg, 9.9 mg, 9.735 mg, 9.57 mg, 9.405 mg, 9.24 mg, 9.075 mg, 8.91 mg, 8.745 mg, 8.58 mg, 8.415 mg, 8.25 mg, 8.085 mg, 7.92 mg, 7.755 mg, 7.59 mg, 7.425 mg, 7.26 mg, 7.095 mg, 6.93 mg, 6.765 mg, 6.6 mg, 6.435 mg, 6.27 mg, 6.105 mg, 5.94 mg, 5.775 mg, 5.61 mg, 5.445 mg, 5.28 mg, 5.115 mg, 4.95 mg, 4.785 mg, 4.62 mg, 4.455 mg, 4.29 mg, 4.125 mg, 3.96 mg, 3.795 mg, 3.63 mg, 3.465 mg, 3.3 mg, 3.135 mg, 2.97 mg, 2.805 mg, 2.64 mg, 2.475 mg, 2.31 mg, 2.145 mg, 1.98 mg, 1.815 mg or 1.65 mg bioavailable iron or heme iron per 100 gram pet food. In another embodiment such amount is 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, or 20 mg bioavailable iron or heme iron per 100 gram pet food.

Example 4 shows that the iron bioavailability of the pet food is higher than corresponding pet foods supplemented with non-heme iron. The animal myoglobins produced here can therefore be used to supplement a plant-based meat substitute with the same level of bioavailable heme iron than the conventional meat product it aims at mimicking, regardless of the presence of inhibitory plant-derived polyphenols.

### General Terms

Unless stated otherwise, all technical and scientific terms used herein have the same meaning as customarily and ordinarily understood by a person of ordinary skill in the art to which this invention belongs, and read in view of this disclosure.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a method as described herein may comprise additional step(s) than the ones specifically identified, said additional step(s) not altering the unique characteristic of the invention. In addition, the verb “to consist” may be replaced by “to consist essentially of” meaning that a pet food, a meat substitute, an isolated myoglobin, a gene construct, a host cell (or methods) as described herein may comprise additional component(s) (or additional steps) than the ones specifically identified, said additional component(s) not altering the unique characteristic of the invention.

Reference to an element by the indefinite article “a” or “an” does not exclude the possibility that more than one of the elements is present, unless the context clearly requires that there be one and only one of the elements. The indefinite article “a” or “an” thus usually means “at least one”. As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The word “about” or “approximately” when used in association with a numerical value (e.g. about 10) preferably means that the value may be the given value (of 10) more or less 0.1% of the value. As used herein, the term “and/or” indicates that one or more of the stated cases may occur, alone or in combination with at least one of the stated cases, up to with all of the stated cases.

In the context of this application, all percentages in the context of a concentration or composition refer to weight percentages, unless defined otherwise. In the context of this application, expressions such as “a parameter having a value of at least X, Y or Z” should be interpreted as said parameter having a value of at least X, of at least Y, or of at least Z.

Various embodiments are described herein. Each embodiment as identified herein may be combined together unless otherwise indicated. All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls. One skilled in the art will recognize many methods and materials similar or equivalent to those described herein, which could be used in the practice of the present invention. Indeed, the present invention is in no way limited to the methods and materials described. The present invention is further described by the following examples which should not be construed as limiting the scope of the invention.

### Sequence Identity

In the context of the invention, a nucleic acid molecule such as a nucleic acid molecule encoding an isolated myoglobin is represented by a nucleic acid or nucleotide sequence which encodes a protein fragment or a polypeptide or a peptide or a derived peptide. It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity with SEQ ID NO: X; or
ii. a nucleotide sequence the sequence of which differs from the sequence of a nucleic acid molecule of (i) due to the degeneracy of the genetic code; or
iii. a nucleotide sequence that encodes an amino acid sequence that has at least 60% amino acid identity or similarity with an amino acid sequence encoded by a nucleotide sequence SEQ ID NO: X.

Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively, percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gin; 11e to Leu or Val; Leu to 11e or Val; Lys to Arg; Gln or Glu;

Met to Leu or 11e; Phe to Met, Leu or Tyr; Ser to Thr; Thr to Ser; Trp to Tyr; Tyr to Trp or Phe; and, Val to 11e or Leu.

### Proteins and Amino Acids

The terms “protein” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (11e) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid, as described herein . . .

### Gene or Coding Sequence

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide. As used herein, a “regulator” or “transcriptional regulator” is a protein that controls the rate of transcription of genetic information from DNA to messenger RNA, by binding to a specific DNA sequence.

### Promoter

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” and/or “repressible” promoter is a promoter that is physiologically or developmentally regulated to be induced and/or repressed, e.g. by the application of a chemical inducer or repressing signal.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. Linking can be accomplished by ligation at convenient restriction sites or at adapters or linkers inserted in lieu thereof, or by gene synthesis.

### Gene Constructs and Expression Vectors

Gene constructs as described herein could be prepared using any cloning and/or recombinant DNA techniques, as known to a person of skill in the art, in which a nucleotide sequence encoding said isolated myoglobin is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

The phrase “expression vector” or “vector” generally refers to a tool in molecular biology used to obtain gene expression in a cell, for example by introducing a nucleotide sequence that is capable of effecting expression of a gene or a coding sequence in a host compatible with such sequences.

An expression vector carries a genome that is able to stabilize and remain episomal in a cell. Within the context of the invention, a cell may mean to encompass a cell used to make the construct or a cell wherein the construct will be administered. Alternatively, a vector is capable of integrating into a cell's genome, for example through homologous recombination or otherwise.

These expression vectors typically include at least suitable promoter sequences and optionally, transcription termination signals. An additional factor necessary or helpful in effecting expression can also be used as described herein. A nucleic acid or DNA or nucleotide sequence encoding a myoglobin is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been described herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334:31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media. Alternatively, a host cell is a cell that is part of a multicellular organism such as a transgenic plant or animal.

For example if a bacterium (preferably E. coli) is used as host cell, the following regulatory regions may be used. A promoter suitable to be used in a bacterium is lac, trp, tac, T7, phoA, ara, xapA, cad, recA, spc, bla, P1 and P2 from rrnB ribosomal RNA operon, PL promoter from phage A. A terminator suitable to be used in a bacterium is lac, trp, tac, T7 (used in example), phoA, ara, xapA, cad, recA, spc, bla, P1 and P2 from rmnB ribosomal RNA operon, PL terminator from phage A. A preferred promoter used is a T7 promoter and/or a preferred terminator is the T7 terminator. A preferred signal peptide for excretion is E. coli Sec-recognition peptide (SecA), E. coli Tet-recognition peptide, E. coli dsbA, E. coli phoA, E. coli pelB, E. coli MBP (maltose binding protein). A marker suitable for E coli is ampicillin. Alternatively, the proBA operon from E. coli strain K12, including its original transcription regulatory elements may be used to facilitate selection without antibiotics.

In another example, if a yeast is used as host cell, the following regulatory regions may be used. A promoter suitable to be used in yeast may be a constitutive promoter. Examples of suitable constitutive promoters include: a glycolytic promoter selected from FBA1, TPI1, PGK1, PYK1, TDH3, ENO2, HXK2, PGI1, PFK1, PFK2, GPM1 gene or a non-glycolytic promoter of the TEF2 gene. A suitable promoter to be used in yeast may be inducible. If the yeast is a Pichia, the methanol inducible promoter AOX1 is preferred. Otherwise the GAL1 promoter (galactose-inducible) may be used when the yeast is S. cerevisiae. The genes mentioned from which a promoter could be derived for a yeast as host cell could also be used to derive a terminator for the same yeast. A preferred signal peptide for excretion for Pichia (and Saccharomyces) includes: the S. cerevisiae alpha mating factor pre-pro-secretion signal peptide, the S. cerevisiae Ost1 signal peptide, the S. cerevisiae Aga2 signal peptide and fusions thereof.

In another example, if a filamentous fungus is used as host cell, the following regulatory regions may be used. The following promoters may be used: the Aspergillus niger glucoamylase promoter (glaA), the Aspergillus nidulans alcohol dehydrogenase promoter (alcA) or the Aspergillus oryzae taka-amylase A promoter (amyB), the Aspergillus niger alcohol dehydrogenase promoter (adhA), the Trichoderma reesei pyruvate kinase promoter (pki) or the Aspergillus nidulas glyceraldehyde-3-phosphate dehydrogenase promoter (gpdA). The genes mentioned from which a promoter could be derived for a filamentous fungus as host cell could also be used to derive a terminator for the same filamentous fungus. A preferred signal peptide for excretion for a filamentous fungus, preferably an Aspergillus includes: the Aspergillus niger glucoamylase signal peptide (glaA), the Aspergillus niger a-galactosidase signal peptide (AgIB) and the Trichoderma reesei cellobiohydrolase I (Cbhl). A preferred promoter and terminator for Aspergillus (more preferably for Aspergillus niger) are the glucoamylase promoter and the glucoamylase terminator of Aspergillus niger.

Gene constructs described herein can be placed in expression vectors. Thus, in another aspect there is provided an expression vector comprising a gene construct as described in any of the preceding embodiments.

Expression may be assessed by any method known to a person of skill in the art. For example, expression may be assessed by measuring the levels of transgene expression in the transduced tissue on the level of the mRNA or the protein by standard assays known to a person of skill in the art, such as qPCR, RNA sequencing, Northern blot analysis, Western blot analysis, mass spectrometry analysis of protein-derived peptides or ELISA. Expression may be assessed at any time after administration of the gene construct, expression vector or composition as described herein. In some embodiments herein, expression may be assessed after 1 week, 2 weeks, 3 weeks, 4, weeks, 5 weeks, 6 weeks, 7 weeks, 8 weeks, 9, weeks, 10 weeks, 11 weeks, 12 weeks, 14 weeks, 16 weeks, 18 weeks, 20 weeks, 22 weeks, 24 weeks, 28 weeks, 32 weeks, 36 weeks, 40 weeks, or more.

Suitable cell culturing methods for use in the process of producing recombinant protein are known to the skilled person, for example, in van′t Riet, K. and Tramper, J., 1st edition, Basic Bioreactor Design, CRC Press, NY, 1991. Such methods include, but are not limited to, submerged fermentation in liquid media, surface fermentation on liquid media and solid-state fermentations. Cell culturing may, for example, be performed by cultivation in micro-titer plates, shake-flasks, small-scale benchtop bioreactors, medium-scale bioreactors and/or large-scale bioreactors in a laboratory and/or an industrial setting. Suitable cell culturing modes include, but are not limited to, continuous, batch and/or fed-batch fermentation as well as their combinations. Cell culturing may be performed using continuous fermentation, batch fermentation, preferably fed batch fermentation.

In the context of the invention, “culture medium”, hereinafter alternately referred to as “growth medium”, can be interpreted to encompass cases wherein the cultured cells are absent as well as cases wherein the cultured cells are present in the culture medium. “Culture broth” refers to the culture medium wherein the cultured cells are present. “Culture supernatant” refers to the culture medium wherein the cultured cells are absent. “Cell-free extract” refers to a cell lysate not comprising the cellular debris. Cell culturing as part of the process of the invention can be performed under conditions conducive to the production of the introduced myoglobins, which are known to the skilled person. Such conditions depend not only on the chemical composition of the culture medium but also on other process parameters including culture duration, temperature, O₂ levels in the culture broth and/or headspace, CO₂ levels in the culture broth and/or headspace, pH, ionic strength, agitation speed, hydrostatic pressure and the like. Cell culturing can take place using a culture medium comprising suitable nutrients, such as carbon and nitrogen sources and additional compounds such as inorganic salts and vitamins, using procedures known in the art (see, e.g. Bennett, W. and Lasure, L., 1^{st} edition, More Gene Manipulations in Fungi, Academic Press, CA, 1991). Suitable growth media are available from commercial suppliers or may be prepared using published compositions that are suitable for the respective hosts (e.g. in catalogues of the Centraalbureau Voor Schimmelcultures collection (CBS) or of the American Type Culture Collection (ATCC)).

The exact composition of the growth medium and the values of culture process parameters are not critical features of the invention. Any growth medium composition may be contemplated, as long as it allows for growth of the host cell and production of the introduced myoglobins. The growth medium will typically comprise a carbon source to be used for the growth of the cultured cell. The skilled person understands that suitable carbon sources may be added externally to the growth medium or may already be present in said medium. Carbon sources may be present or added individually or in mixtures of multiple carbon sources. Examples of suitable carbon sources known in the art include simple sugars such as glucose, glycerol, maltose, sucrose, xylose, arabinose, complex sugars such as maltodextrins, hydrolysed starch, starch, molasses, and second-generation feedstocks. Second-generation feedstocks can be particularly attractive because of their lower carbon footprint. Second-generation feedstocks will typically comprise lignocellulosic material. Such material includes any lignocellulose and/or hemicellulose-based materials. Such material may be sourced from agricultural, industrial or municipal, preferably agricultural, waste streams. Examples of suitable materials include (agricultural) biomass, commercial organic matter, municipal solid waste, virgin biomass such as waste paper and garden waste, or non-virgin biomass. General forms of biomass include trees, shrubs and pastures, wheat, wheat straw, sugarcane bagasse, switchgrass, Japanese pampas grass, corn, corn stover, corn cob, canola stalk, soybean stalk, sweet corn, corn kernels, products and by-products from cereal milling (including wet milling and dry milling), such as corn, wheat, and barley, often referred to as “bran or fiber”, and municipal solids. Biomass can also be grassy materials, agricultural residues, forestry residues, municipal solid waste, waste paper, and pulp and paper mill residues. Agricultural biomass includes branches, shrubs, tows, corn and corn straw, energy crops, forests, fruits, flowers, cereals, pastures, herbaceous crops, leaves, bark, needles, logs, roots, young trees, short term rotating woody crops, shrubs, switch herbs, trees, vegetables, fruits, vines, sugar beet pulp, wheat middlings, oat hulls, and hard and soft timber (not including toxic wood), and organic waste materials resulting from agricultural processes including agriculture and forestry activities, particularly forestry wood waste. Agricultural biomass may be any of the foregoing alone, or any combination or mixture thereof. Carbon sources such as organic acids, aldehydes, ketones, esters and alcohols may also be contemplated. The use of growth media comprising combinations of multiple different carbon sources may also be contemplated in the process of the invention. Such media could, as a non-limiting example, combine more oxidized carbon sources such as organic acids with more reduced carbon sources such as alcohols. Examples of suitable nitrogen sources known in the art include soy bean meal, corn steep liquor, yeast extract, whey protein, egg protein, casein hydrolysate, urea, ammonia, ammonium salts and nitrate salts. Examples of additional suitable compounds known in the art include phosphate, sulphate, metals such as magnesium, trace elements and vitamins. The exact growth medium requirements will vary based on the host cell, e.g. between yeasts, bacteria and filamentous fungi, said requirements will be known to the skilled person. Accordingly, the growth medium may be a complete (rich) medium or a minimal medium, i.e. a medium comprising only the absolutely necessary components for growth depending on the cultured host cell.

Similar to the composition of the growth medium, process parameters can be assigned any value, as long as they allow for growth of the host cell and production of the introduced myoglobins. Typically, said values will differ based on the host cell that is being cultured and will be known to the skilled person. Preferably, the process according to the invention is an oxygen-limited or aerobic process, meaning that cell culturing is performed under oxygen-limited or aerobic conditions, more preferably the process is oxygen-limited. Oxygen-limited conditions, also known as a micro-aerobic conditions, are culture conditions in which the oxygen consumption is limited by the availability of oxygen. The degree of oxygen limitation is determined by the amount and composition of the ingoing gas flow as well as the actual mixing/mass transfer properties of the fermentation equipment used. Preferably, under oxygen-limited conditions in a liquid culture, the rate of oxygen consumption is at least about 5.5 mmol/L/h, more preferably at least about 6 mmol/L/h and even more preferably at least about 7 mmol/L/h. Aerobic conditions are culture conditions in which the oxygen consumption is not limited by the availability of oxygen.

Cell culturing may be performed at a temperature value that is optimal for the cell, typically at a temperature range of 16-42° C. In some embodiments, the temperature ranges between 20-40° C., more preferably between 25-38° C., most preferably between 28-36° C. In some most preferred embodiments, a temperature value of about 30 or 36° C. is used.

Cell culturing may be performed at a pH value that is optimal for the cell. In some embodiments, the culture pH value is about pH 2.5, about pH 3.0, about pH 3.5, about pH 4.0, about pH 4.5, about pH 5, about pH 5.5, about pH 6, about pH 6.5, about pH 7, about pH 7.5, about pH 8.0, about pH 8.5, about pH 9. In preferred embodiments, the pH ranges from about pH 3.0 to about pH 9, more preferably from about pH 3.5 to pH 7. In some most preferred embodiments, a pH value of about 6 is used.

Cell culturing may be performed at an ionic strength value of the culture medium that is optimal for the cell, typically at a range between 50 mM-2 M. In some embodiments, the ionic strength of the culture medium ranges between 75 mM-1 M, more preferably between 100 mM-750 mM. In some most preferred embodiments, an ionic strength value of about 100 mM is used.

### Overview of the Sequence Listing

| | SEQ ID NO | SEQ ID NO |
|---|---|---|
| Protein | protein | coding nucleic acid |
| Rat myoglobin | 1 | 7 |
| Rabbit myoglobin | 2 | 8 |
| Mouse myoglobin | 3 | 9 |
| Steppe mammoth myoglobin | 4 | 10 |
| Woolly mammoth myoglobin | 5 | 11 |
| Tuna myoglobin | 6 | 12 |
| Woolly mammoth κ-casein | 13 | |
| Woolly mammoth β-casein | 14 | |
| Cattle myoglobin | 15 | |

Detailed sequences

-SEQ ID NO: 1 Rat myoglobin
MGLSDGEWQMVLNIWGKVEGDLAGHGQEVLISLFKAHPETLEKFDKFKNLKSEEEMKSSED
LKKHGCTVLTALGTILKKKGQHAAEIQPLAQSHATKHKIPVKYLEFISEVIIQVLKKRYSGDFGA
DAQGAMSKALELFRNDIAAKYKELGFQG
-SEQ ID NO: 2 Rabbit myoglobin
MGLSDAEWQLVLNVWGKVEADLAGHGQEVLIRLFHTHPETLEKFDKFKHLKSEDEMKASED
LKKHGNTVLTALGAILKKKGHHEAEIKPLAQSHATKHKIPVKYLEFISEAIIHVLHSKHPGDFGA
DAQAAMSKALELFRNDIAAQYKELGFQG
-SEQ ID NO: 3 Mouse myoglobin
MGLSDGEWQLVLNVWGKVEADLAGHGQEVLIGLFKTHPETLDKFDKFKNLKSEEDMKGSED
LKKHGCTVLTALGTILKKKGQHAAEIQPLAQSHATKHKIPVKYLEFISEIIIEVLKKRHSGDFGAD
AQGAMSKALELFRNDIAAKYKELGFQG
-SEQ ID NO: 4 Steppe mammoth myoglobin
MGLSDGEWELVLKTWGKVEADIPGHGLEVFVRLFTGHPETLEKFDKFKHLKTEGEMKASED
LKKQGVTVLTALGGILKKKGHHQAEIQPLAHSHATKHKIPIKYLEFISDAIIHVLQSKHPAEFGA
DAQGAMKKALELFRNDIAAKYKELGFQG
-SEQ ID NO: 5 Woolly mammoth myoglobin
MGLSDGEWELVLKTWGKVEADIPGHGLEVFVRLFTGHPETLEKFDKFKHLKTEGEMKASED
LKKQGVTVLTALGGILKKKGHHQAEIQPLAQSHATKHKIPIKYLEFISDAIIHVLQSKHPAEFGA
DAQGAMKKALELFRNDIAAKYKELGFQG
-SEQ ID NO: 6 Tuna myoglobin
MADFDAVLKCWGPVEADYTTIGGLVLTRLFKEHPETQKLFPKFAGIAQADIAGNAAVSAHGA
TVLKKLGELLKAKGSHAAILKPLANSHATKHKIPINNFKLISEVLVKVMHEKAGLDAGGQTALR
NVMGIIIADLEANYKELGFSG
-SEQ ID NO: 7 Rat myoglobin nucleic acid
ATGGGCCTGAGCGATGGCGAATGGCAGATGGTGCTGAACATTTGGGGCAAAGTGGAAG
GCGATCTGGCGGGCCATGGCCAGGAAGTGCTGATTAGCCTGTTTAAAGCGCATCCGGA
AACCCTGGAAAAATTTGATAAATTTAAAAACCTGAAAAGCGAAGAAGAAATGAAAAGCAG
CGAAGATCTGAAAAAACATGGCTGCACCGTGCTGACCGCGCTGGGCACCATTCTGAAAA
AAAAAGGCCAGCATGCGGCGGAAATTCAGCCGCTGGCGCAGAGCCATGCGACCAAACA
TAAAATTCCGGTGAAATATCTGGAATTTATTAGCGAAGTGATTATTCAGGTGCTGAAAAAA
CGCTATAGCGGCGATTTTGGCGCGGATGCGCAGGGCGCGATGAGCAAAGCGCTGGAAC
TGTTTCGCAACGATATTGCGGCGAAATATAAAGAACTGGGCTTTCAGGGC
-SEQ ID NO: 8 Rabbit myoglobin nucleic acid
ATGGGCCTGAGCGATGCGGAATGGCAGCTGGTGCTGAACGTGTGGGGCAAAGTGGAAG
CGGATCTGGCGGGCCATGGCCAGGAAGTGCTGATTCGCCTGTTTCATACCCATCCGGAA
ACCCTGGAAAAATTTGATAAATTTAAACATCTGAAAAGCGAAGATGAAATGAAAGCGAGC
GAAGATCTGAAAAAACATGGCAACACCGTGCTGACCGCGCTGGGCGCGATTCTGAAAAA
AAAAGGCCATCATGAAGCGGAAATTAAACCGCTGGCGCAGAGCCATGCGACCAAACATA
AAATTCCGGTGAAATATCTGGAATTTATTAGCGAAGCGATTATTCATGTGCTGCATAGCAA
ACATCCGGGCGATTTTGGCGCGGATGCGCAGGCGGCGATGAGCAAAGCGCTGGAACTG
TTTCGCAACGATATTGCGGCGCAGTATAAAGAACTGGGCTTTCAGGGC
-SEQ ID NO: 9 Mouse myoglobin nucleic acid
ATGGGCCTGAGCGATGGCGAATGGCAGCTGGTGCTGAACGTGTGGGGCAAAGTGGAAG
CGGATCTGGCGGGCCATGGCCAGGAAGTGCTGATTGGCCTGTTTAAAACCCATCCGGA
AACCCTGGATAAATTTGATAAATTTAAAAACCTGAAAAGCGAAGAAGATATGAAAGGCAG
CGAAGATCTGAAAAAACATGGCTGCACCGTGCTGACCGCGCTGGGCACCATTCTGAAAA
AAAAAGGCCAGCATGCGGCGGAAATTCAGCCGCTGGCGCAGAGCCATGCGACCAAACA
TAAAATTCCGGTGAAATATCTGGAATTTATTAGCGAAATTATTATTGAAGTGCTGAAAAAA
CGCCATAGCGGCGATTTTGGCGCGGATGCGCAGGGCGCGATGAGCAAAGCGCTGGAA
CTGTTTCGCAACGATATTGCGGCGAAATATAAAGAACTGGGCTTTCAGGGC
-SEQ ID NO: 10 Steppe mammoth myoglobin nucleic acid
ATGGGACTCAGCGACGGGGAATGGGAGTTGGTGTTGAAAACCTGGGGGAAAGTGGAGG
CTGACATCCCGGGCCATGGGCTGGAAGTCTTCGTCAGGCTCTTCACAGGTCATCCCGAG
ACCCTGGAGAAGTTCGACAAGTTTAAGCACCTGAAGACAGAGGGCGAGATGAAGGCCTC
CGAGGACCTGAAGAAGCAAGGTGTTACTGTGCTCACTGCCCTGGGGGGCATCCTCAAG
AAGAAAGGGCATCACCAGGCGGAGATTCAGCCCCTGGCCCATTCTCATGCCACCAAGCA
CAAGATCCCCATCAAGTATCTGGAGTTCATCTCGGACGCCATCATCCACGTTCTGCAAAG
CAAGCATCCTGCGGAATTTGGCGCTGATGCCCAGGGAGCCATGAAAAAGGCCTTGGAG
CTGTTCCGGAATGACATTGCGGCCAAGTATAAGGAGCTGGGGTTCCAGGGCTAG
-SEQ ID NO: 11 Woolly mammoth myoglobin nucleic acid
ATGGGACTCAGCGACGGGGAATGGGAGTTGGTGTTGAAAACCTGGGGGAAAGTGGAGG
CTGACATCCCGGGCCATGGGCTGGAAGTCTTCGTCAGGCTCTTCACAGGTCATCCCGAG
ACCCTGGAGAAGTTCGACAAGTTTAAGCACCTGAAGACAGAGGGCGAGATGAAGGCCTC
CGAGGACCTGAAGAAGCAAGGTGTTACTGTGCTCACTGCCCTGGGGGGCATCCTCAAG
AAGAAAGGGCATCACCAGGCGGAGATTCAGCCCCTGGCCCAGTCTCATGCCACCAAGC
ACAAGATCCCCATCAAGTATCTGGAGTTCATCTCGGACGCCATCATCCACGTTCTGCAAA
GCAAGCATCCTGCGGAATTTGGCGCTGATGCCCAGGGAGCCATGAAAAAGGCCTTGGA
GCTGTTCCGGAATGACATTGCGGCCAAGTATAAGGAGCTGGGGTTCCAGGGCTAG
SEQ ID NO: 12 Tuna myoglobin nucleic acid
ATGGCTGACTTTGATGCAGTTCTGAAGTGTTGGGGTCCAGTGGAGGCGGACTACACCAC
CATTGGAGGCCTGGTTCTGACCCGTTTATTCAAAGAGCACCCTGAGACCCAGAAGCTGT
TCCCCAAATTCGCTGGCATCGCCCAGGCTGACATAGCCGGTAACGCAGCTGTTTCTGCT
CATGGTGCCACTGTGCTGAAGAAACTTGGAGAGCTGCTGAAGGCCAAAGGCAGTCACG
CTGCCATCCTAAAACCACTGGCAAACAGCCATGCCACTAAGCACAAGATTCCCATTAATA
ACTTCAAGCTGATTTCTGAGGTCCTTGTGAAGGTCATGCATGAGAAGGCAGGACTCGAT
GCCGGTGGGCAGACAGCCCTGAGGAACGTGATGGGTATCATCATCGCCGACCTTGAGG
CCAACTACAAAGAGCTGGGCTTCTCTGGCTGA
SEQ ID NO: 13 Woolly mammoth K-casein
MKGFLLVVNILLLPLFLAAEVQNQEESRCLEKDERWFCQKAVKYIPNDYVLKSYYRYEPNYN
QFRAAVPINNPYLIYLYPAKQVAVRPHTQIQWQVPSNIYPSPSVPHTYLKPPFIIPPKKTQDKPI
IPPTGTVASIEATVEPKVNTVVNAEASSEFIATNTPEATTVPVISPQI
SEQ ID NO: 14 Woolly mammoth B-casein
MKVFILACLVAFALGRETVENLSSSEIRQFYSEQKPEGVKHEEQQREDEHQNKIQPLFQPQP
LVYPFAEPIPYTVFPPNAIPLAQPIVVLPFPQPEVQLPEAKEITFPRQKLMSFLKSPVMPFFDP
QPNLGTDLENLHLPLPLLQPLRHQLHQPLAQTPVLPLPLSLPKVLPVPQQVIPYPQRGRPIQN
LLYEEPLLDPTRKIYPVAQPLAPVYNPVAYMIGIPCCSTLLTYLHQSSRSQYPIQNKLGYLIAM
PKKVRPT

### LEGEND TO THE FIGURES

FIG. 1. Comparison of the myoglobin protein sequence from six animal species. The sequence alignment shows sequences from tuna (Thunnus orientalis, SEQ ID NO: 6), the steppe mammoth (Mammuthus trogontherii, SEQ ID NO: 4), rat (Rattus norvegicus, SEQ ID NO: 1), mouse (Mus musculus, SEQ ID NO: 3), rabbit (Oryctolagus cuniculus, SEQ ID NO: 2) and cattle (Bos taurus, SEQ ID NO: 15). The level of amino acid conservation is indicated by shading, using the BLOSUM62 score.

FIG. 2. Tridimensional structure of myoglobin from 6 animal species. Predicted structure of the wildtype myoglobin from rabbit (Oryctolagus cuniculus, SEQ ID NO: 2), mouse (Mus musculus, SEQ ID NO: 3), rat (Rattus norvegicus, SEQ ID NO: 1), the steppe mammoth (Mammuthus trogontherii, SEQ ID NO: 4), tuna (Thunnus orientalis, SEQ ID NO: 6), and cattle (Bos taurus, SEQ ID NO: 15), shown as ribbon diagrams. Heme is shown in black.

FIG. 3. Expression vectors for animal myoglobin production in Pichia pastoris. Six vectors, carrying the codon-optimized sequence encoding the 6 myoglobins listed above, have been generated. The expression cassette is produced by restriction using the two BgIII cut sites, allowing genome integration by recombination between chromosomal DNA and the AOX1 promoter on the one hand, and the AOX1 3′ fragment on the other hand.

FIG. 4. Confirmation of the presence of the myoglobin expression cassette in Pichia pastoris. Picture of an agarose gel after electrophoresis of a PCR product, obtained after amplification from genomic DNA of different yeast colonies (up to 8 per myoglobin construct) using primers that are specific for the myoglobin expression cassette.

FIG. 5. Production of extracellular animal myoglobin (Mb) in Pichia pastoris. Cell-free supernatants were collected after fermentation with methanol induction in a 96 well plate format and analyzed by SDS-PAGE, revealed a protein of the expected molecular weight for myoglobin. Clones are shown that express myoglobin either from cattle, the steppe mammoth, tuna or rabbit. Commercially available myoglobin purified from horse heart muscle was used as standard.

FIG. 6. Matrix of a plant-based wet pet food. A plant-based matrix for a wet pet food product was prepared either without (A) or with (B) myoglobin 0.1% w/w.

FIG. 7. Color stability of myoglobin obtained by precision fermentation in Pichia pastoris. Colorimetric ΔE values were calculated for three independent myoglobin samples (labelled A, B and C) over a 2-month storage period at 4° C.

FIG. 8. Color effect of different animal myoglobins (Mb) in plant-based wet pet food. Raw and heat-sterilized samples were prepared without myoglobin or with 0.1% w/w of either rabbit or cattle myoglobin obtained by precision fermentation in Pichia pastoris. (A) Samples were imaged at day 0. (B) The color effect of myoglobin in plant-based wet-pet food was quantified. The bar graph shows colorimetric L*a*b* values of each sample.

FIG. 9. Color and color stability effect of myoglobin in plant-based wet pet food. Raw and heat-sterilized samples prepared with different concentrations of myoglobin obtained by precision fermentation in Pichia pastoris (0, 0.2, 0.5, 1% w/w) were imaged (A) at day 0 and (B) after 20 days of storage at 4° C. (C) Colorimetric L*a*b* values of each sample at day 0. (D) Relative color difference (ΔE) after 20 days of storage at 4° C. Data are expressed as relative values compared to the corresponding raw or sterilized samples without myoglobin.

FIG. 10. Plant-based wet pet food containing cattle or rabbit myoglobin obtained by precision fermentation in Pichia pastoris. The plant-based wet pet food was prepared without myoglobin, with 0.1% cattle myoglobin or with 0.1% rabbit myoglobin.

FIG. 11. Aromatic profile of raw and heat-sterilized plant-based wet pet food with animal myoglobin obtained by precision fermentation in Pichia pastoris. (A) Distribution of the different classes of volatile organic compounds detected by SPME-GC-MS, for raw and heat-sterilized plant-based wet pet food without myoglobin or with 0.1% w/w myoglobin obtained by precision fermentation in Pichia pastoris. Data is expressed as relative value. The proportion of pyrazines is indicated at the top of each bar. (B) Principal component analysis (PCA) score and loading plot of PC1 and PC2 for volatile organic compounds emitted by raw and heat-sterilized plant-based wet pet food without myoglobin or with 0.1% w/w myoglobin. Selected volatile organic compounds (loadings) are depicted by an arrow, and the corresponding compound name is listed on the right side of the graph. (C) Correlation loadings of PC1 and PC2 for volatile organic compounds (n=111) emitted by raw and heat-sterilized plant-based wet pet food without myoglobin and with 0.1% w/w myoglobin.

FIG. 12. Conventional meat-associated volatile organic compounds and relation with the addition of myoglobin to plant-based wet pet food. (A) Detected levels (peak surface area) of selected volatile organic compounds in raw and heat-sterilized plant-based wet pet food without myoglobin or with 0.1% w/w myoglobin obtained by precision fermentation in Pichia pastoris. Asterisks represent statistically significant differences (t-test) between the pairs of samples: (*)=p<0.05, (***)=p<0.001. (B) Aroma description (The Good Scent Company information system) of the selected volatile organic compounds, which are also found in cooked conventional meat [1-4].

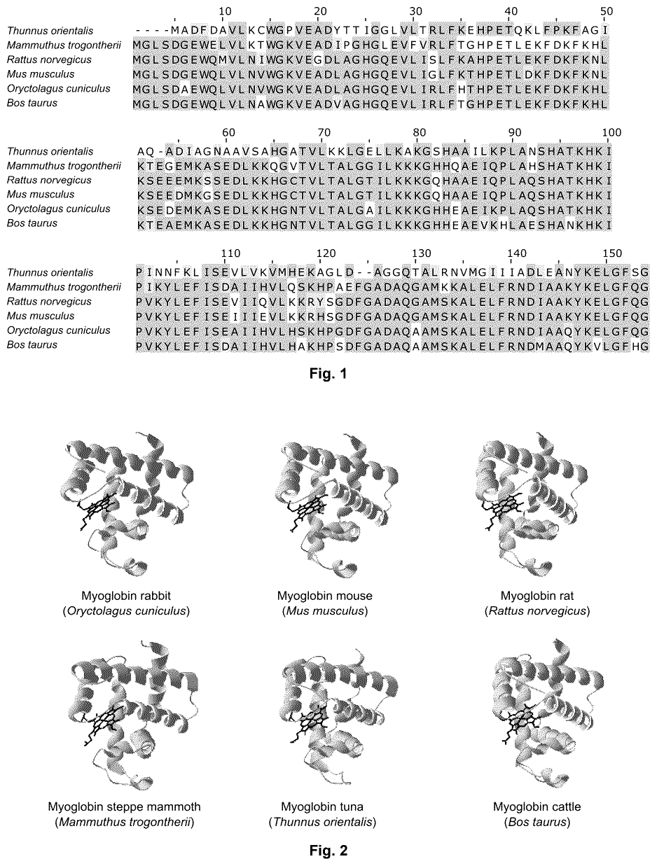

### EXAMPLES

By way of example and not limitation, the following non-limiting examples are illustrative of various embodiments provided by the present disclosure.

### Example 1: Selection of Target Proteins

Myoglobin (Mb) is a relatively small globular protein of about 17 kD, found in heart and skeletal muscles. It carries a single heme group capable of reversible oxygen binding (FIG. 1), allowing myoglobin to transport oxygen from the cell surface to mitochondria. Mb is also the primary pigment responsible for meat color. In oxygenated Mb (oxymyoglobin), the iron atom of the heme group is coordinated by four nitrogen atoms of the porphyrin ring, the so-called “proximal” histidine (His93) in the Mb chain, and an oxygen molecule. This can be further stabilized by hydrogen bonding between the oxygen molecule and another histidine in the heme binding pocket of Mb, the “distal” histidine (His64). Under this form, Mb has a typical bright red color. In the absence of oxygen, Mb takes a darker red color (deoxymyoglobin). When the heme iron is oxidized from the ferrous (Fe(II)) to the ferric (Fe(III)) state, it is unable to bind oxygen and Mb displays a brown color (metmyoglobin), as seen in cooked meat. Heme iron oxidation also reduces the affinity of Mb for heme, leading to increased heme loss and subsequent unfolding of the protein [5].

Since myoglobin is present in the heart and skeletal muscles of vertebrates, it is also found in meat and fish products at a concentration that varies depending on the species, the type of muscle tissue and the age of the animal. Reported concentrations of myoglobin in muscle tissue from various animal species are listed in Table 1 below.

TABLE 1
Myoglobin concentration in muscle tissue from six animal species.
Reported myoglobin concentrations are indicated in % (w/w of
muscle wet mass). Data from the Asian elephant are used here
as a proxy for the steppe mammoth, an extinct species from
which no muscle tissue sample is available for analysis.

| Species | Myoglobin concentration | Reference |
|---|---|---|
| Cattle | 0.243% | [6] |
| | 0.199 to 0.364% | [7] |
| | 0.1 to 2% | [8] |
| | 0.2 to 1.8% | [9] |
| Tuna | 0.037 to 2.44% | [10] |
| | 0.0193% | [11] |
| | 0.965% | [12] |
| | 0.023 to 0.157% | [13] |
| | 0.13 to 2.37% | [14] |
| Rabbit | 0.02% | [15] |
| | 0.057 to 0.072% | [16] |
| | 0.08% | [17] |
| | 0.04% | [18] |
| Mouse | 0.000149 to 0.00023% | [19] |
| Rat | 0.07 to 0.12% | [20] |
| Asian elephant | 0.46% | [21] |

The amino acid sequence of Mb has been strongly conserved during evolution and shows relatively little variation between species (FIG. 1). Yet single amino acid changes have been reported that strongly affect the relative affinity of Mb for dioxygen and carbon dioxide, its auto-oxidation rate or propensity to aggregate [22]. Here, when screening for candidate Mb to be produced as ingredient for meat substitute as pet food, we carefully examined the sequence of various vertebrate species. We included extant species whose muscle tissue is commonly consumed for their meat, as well as an extinct Elephantidae, i.e. the steppe mammoth (Mammuthus trongotherii) [23]. We previously described a relevant substitution in the myoglobin sequence of this species: a histidine at position 92 instead of a glutamine (His92) (FIG. 1), that confer it a higher positive surface charge (net surface charge (Z_{Mb}) at pH 6.5=2.67, compared to e.g. myoglobin from the Asian elephant with Z_{Mb} at pH 6.5=2.11) [24]. This is reminiscent of adaptation of Mb to deep-sea diving in cetaceans, where increased positive surface charge led to decreased attractive interactions between Mb molecules at a contact distance, increasing Mb stability and preventing its aggregation [25,26]. Overall, Mb from the steppe mammoth presents several characteristics that make it particularly attractive as an ingredient in meat substitutes. Here, we evaluate the production and characteristics of recombinant Mb from the steppe mammoth and from cattle (Bos taurus), the latter serving as a reference since this extant species is widely consumed for its red meat. In addition, we reasoned that pets would be less likely to suffer adverse food reactions (AFRs) from eating myoglobin from animals they are evolutionarily adapted to consume. For this reason, we also considered myoglobin from species known to be the preys of the ancestors of modern pets, i.e. rabbit (Oryctolagus cuniculus) as the prey of wolves and the first domesticated dogs [27], house mouse (Mus musculus) or rat (Rattus norvegicus) as preys of the first domesticated cats [28]. Finally, we also examined myoglobin from tuna (Thunnus orientalis) as an intensively exploited fish with a particularly high concentration of myoglobin in the muscle (Table 1), and that is very attractive for cats [29].

### Example 2: Extracellular Production of Myoglobin in Pichia pastoris

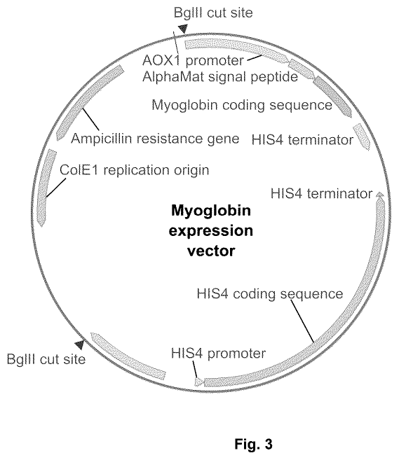

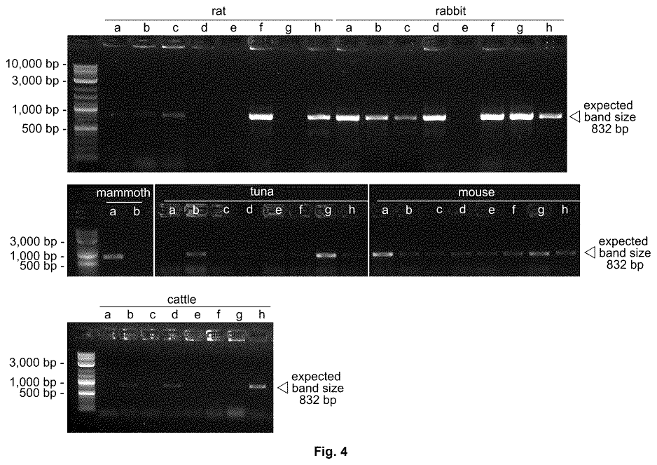

The sequences coding for Mb from the steppe mammoth, cattle, rat rabbit, mouse and tuna (FIG. 1, 2) were cloned downstream of the methanol-inducible AOX1 promoter of Pichia pastoris, and upstream of a histidine prototrophy selection marker and the AOX1 terminator (FIG. 3). Myoglobin is naturally found in the cytoplasm of muscle cells. To facilitate recombinant protein purification, we fused a sequence encoding a signal peptide in frame with the myoglobin coding sequence, in order to target nascent Mb proteins to the secretory pathway for excretion outside the cell (FIG. 3). These constructs were used to transform histidine-auxotroph (his4) Pichia pastoris cells, and transformants were selected for their ability to grow in the absence of histidine. The presence of the myoglobin expression cassette in the genome of the histidine prototroph clones was then verified by PCR (FIG. 4).

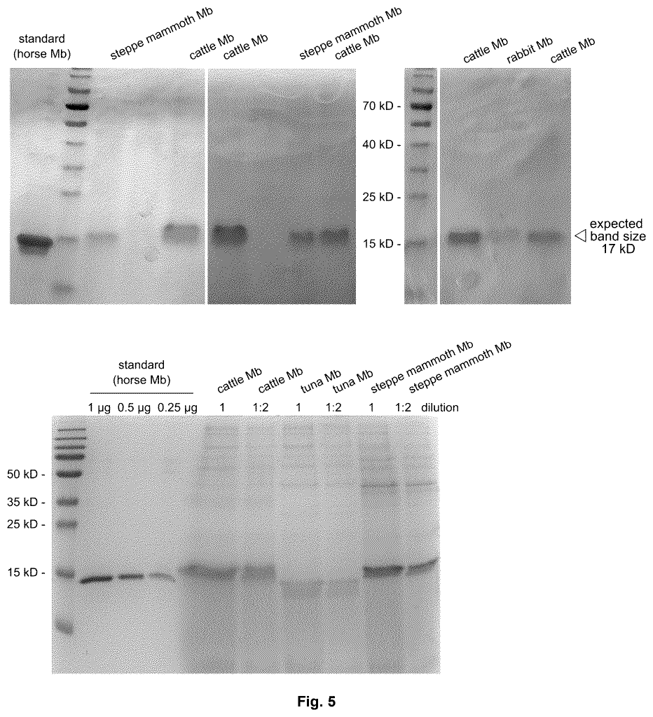

For each construct, verified transformants were then tested for their ability to produce extracellular animal myoglobin after methanol induction in 96 wells microplates. The presence of a methanol-inducible protein of the expected molecular weight was confirmed after protein electrophoresis and staining with Coomassie blue (FIG. 5).

In order to obtain larger amount of material for the preparation of plant-based meat substitutes, fermentation runs were conducted in a 10 L fermenter. The cell-free supernatant was purified and concentrated by ultrafiltration, to obtain a concentrated myoglobin solution that also contains residual yeast proteins. The observed protein purity of myoglobin in the final product is typically in the range of 75-85%. Since the yeast that is used as a production host, Pichia pastoris, has an established history of safe use in food production, e.g. for multiple Generally Recognized As Safe (GRAS) products in the United States, and has been granted Qualified Presumption of Safety (QPS)

in the European Union [30], consumption of Pichia proteins does not pose any safety issue. On the other hand, the presence of yeast proteins might even contribute to the palatability of the plant-based meat substitute containing the myoglobin ingredient [31,32].

### Example 3: Preparation of Plant-Based Pet Food

Commercial pet foods have long been based on meat and meat-derived by-products because these products are usually highly palatable to pet mammals, including dogs and cats. A move toward more animal and environmentally friendly food production, and convenient delivery formats for commercial pet foods (e.g. cereal-based kibble) have influenced an increase in the popularity and availability of plant-based pet food. The dominant proteins in such plant-based pet foods have historically been derived from soy, corn, and wheat (gluten). Recently, additional plant proteins have become available, including pea, potato, and rice protein. Since palatability is one of the main criteria in pet-diet decision making [33], producers are developing various strategies to impart desirable “meat-like” properties to their pet food product.

Here we describe the development of a model plant-based pet food in which the properties of animal myoglobin produced in Pichia pastoris can be evaluated. Two main types of wet pet food products are distinguished, ‘pate or loaf’ and ‘chunk and gravy’. We selected pate, which is typically prepared by processing a mixture of edible components under heat to produce a homogeneous semi-solid mass that is structured by heat-coagulated protein. This homogeneous mass is usually packaged into single serve or multi serve packaging which is then sealed and sterilized. Upon packing, the homogeneous mass assumes the shape of the container. Incorporating Maillard components into a sterilized wet pet food product containing restructured meat has been shown to significantly increase its palatability [34].

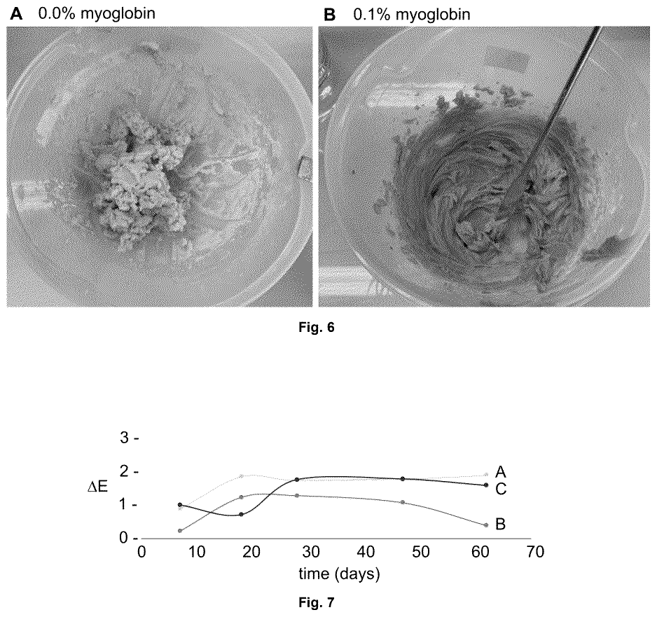

To develop our model plant-based pet food (Table 2, FIG. 6), we formulated a matrix resembling commercial wet pet food in the form of ‘pate’, undergoing similar processing conditions as described above, as laboratory conditions permit.

TABLE 2
Composition of the model plant-based meat substitute compared
to conventional pet food products. The nutritional composition
of the model plant-based pet food product developed here
is compared to that of commercial pet food products, as
described in [35] and [36].

| | | Plant-based pet food - |
|---|---|---|
| Nutrient (g per 100 g) | Commercial pet food | this study |
| Protein (min) | 18-25^{a} | 20 |
| Fat (min) | 5.5-8.5^{a} | 10 |
| Fiber (max) | 1.5^{b} | N/A |
| Moisture (max) | 78^{b} | 70 (water added) |

We then added animal myoglobin obtained by precision fermentation in this pet food matrix, and investigated the influence of myoglobin addition on the aromatic profile, color and texture of plant-based wet pet food.

### Example 4: Nutritional Value

Although pet food producers must pay special attention to ensure adequate intake of certain nutrients such as proteins, amino acids (e.g., taurine, carnitine, methionine, lysine, and tryptophan), vitamins (e.g., Vitamins A, B3, B9, and B12), minerals (e.g., calcium, iron, zinc, and copper), and fats [37], it is important to remember that all species require specific nutrients, rather than specific ingredients. One study based on blood samples analyses showed that only two out of 20 dogs that were fed vegan diets had any deficiencies. Parameters where there were significant differences between groups fed with meat or vegan diets were iron, vitamin B12 and folic acid, though these elements did not move outside of the reference ranges [38].

Conventional meat products constitute an important source of dietary iron. Animal tissue is indeed a source of heme iron, i.e. iron coordinated to a porphyrin (Table 3), while plants and pulses contain non-heme iron [39].

TABLE 3
Myoglobin-bound, heme iron concentration in muscle tissue from
six animal species. The concentration of heme iron bound to
myoglobin in muscle tissue (indicated in mg per 100 gram) has
been calculated from the reported myoglobin concentrations
listed in Table 1, based on a molar mass of 616.5 Da for heme
iron the molecular weight of the corresponding myoglobin.

| | Heme iron concentration |
|---|---|
| Species | in mg/100 g |
| Cattle | 3.51 to 70.2 |
| Tuna | 0.74 to 93.45 |
| Rabbit | 0.70 to 2.76 |
| Mouse | 0.0052 to 0.0080 |
| Rat | 2.43 to 4.17 |
| Asian elephant | 15.96 |

The bioavailability of iron from heme iron sources is 2— to 7-fold higher than the bioavailability of non-heme iron [40]. An additional challenge when formulating plant-based food is that the absorption of non-heme iron is inhibited by phytic acid (myo-inositol hexaphosphate), found in cereals, legumes nuts and seeds, and by polyphenol compounds, which are abundant in fruits and vegetables [39].

We previously demonstrated that the heme iron present in animal myoglobins obtained by precision fermentation in the yeast Pichia pastoris shows the same bioavailability as heme iron present in conventional beef meat [24]. The animal myoglobins produced here can therefore be used to supplement a plant-based meat substitute with the same level of bioavailable heme iron than the conventional meat product it aims at mimicking, regardless of the presence of inhibitory plant-derived polyphenols.

### Example 5: Color and Color Stability of Recombinant Myoglobin

Consumers routinely use product color and appearance to select or reject products [41]. From a physical point of view, the color appearance of a material represents the response of retina rods and cones to the reflected radiation in the so-called ‘visible region’ of the electromagnetic spectrum, in the range between 400 and 700 nm. From a chemico-physical point of view, color is the result of an interaction between a light source and pigments, by which energy is absorbed and emitted as complementary not-absorbed radiations in the visible region [42].

Meat products are characterized by their red color, which is largely imparted by myoglobin [43]. To mimic a meat-like appearance, different food colorants are added to meat substitute products on the market, such as beet juice extract, pomegranate syrup, soy leghemoglobin, carrot juice extract, and lycopene [44].

The surface color of meat and meat analogues is routinely measured with colorimeters which are equipped with a pulsed xenon arc lamp, which illuminates (with D65 being the standard illuminant) the sample with a standardized light beam. The light reflected from the sample surface is then collected using photocells and is used to calculate the coordinates in the color space [45].

Here we have followed the colorimetric stability of independent myoglobin samples obtained by precision fermentation in Pichia pastoris, stored at 4° C. away from light during 2 months. In all three samples, the observed color difference (ΔE) did not reach or surpass 2 at any time point (FIG. 7), indicating that no recognizable color change was observed [46].

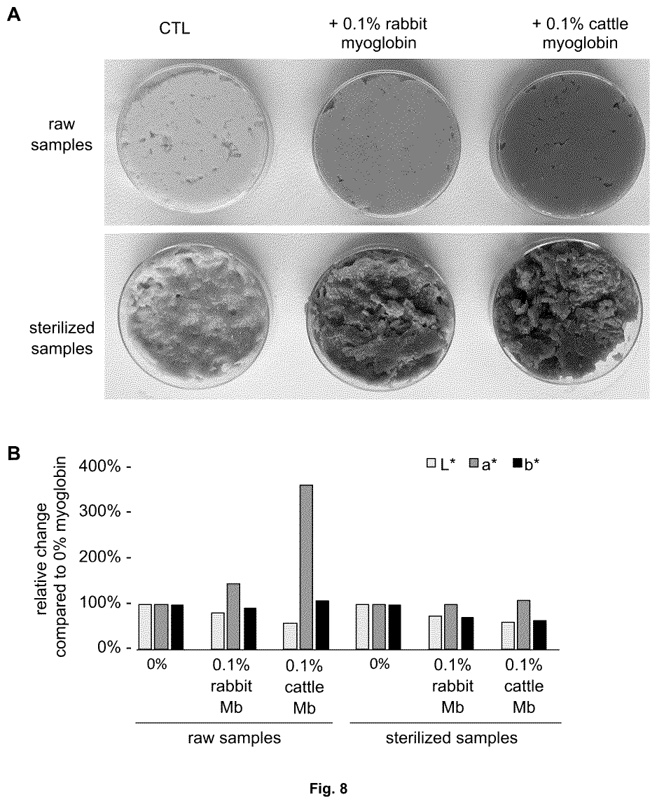

To test the color effect of different animal myoglobins in a plant-based matrix. We first assessed the addition of rabbit and cattle myoglobin produced by precision fermentation in Pichia pastoris (0.1% w/w) to a plant-based wet pet food matrix. We then examined the color of the resulting products either raw or right after heat sterilization (FIG. 8A). In raw products, the lightness (L*) decreased while its redness (a*) increased for both rabbit and cattle myoglobin (FIG. 8B). The increase in redness was more pronounced for the cattle myoglobin. Hernandez et al. (2016) previously measured L*, a* and b* values of beef (Longissimus lumyoglobinorum) samples [47]. The L* value we measured here for raw samples with 0.1% cattle myoglobin (42.9) is in line with the reported L* value for raw beef meat (37.61) found in this previous study [47]. Kozioł et al. measured color changes of rabbit meat during the aging process. The L* value measured for raw rabbit meat 45 minutes post mortem was 59.30 [48]. Our measured L* value for raw samples with 0.1% rabbit myoglobin was 60.27, in agreement with the author's findings. Lightness also similarly decreased in the heat-sterilized products containing myoglobin (FIG. 8B).

Regarding redness, a* values in raw samples were more than two-fold greater in samples containing cattle myoglobin than in rabbit myoblobin (7.56 vs. 3.01, respectively). After baking, the samples showed similar values (7.02 and 6.42 for cattle and rabbit myoglobin, respectively).

Hernandez et al. measured an a* value of 14.42 on raw beef samples [47], a higher value that is likely explained by the low cattle myoglobine concentration in our samples compared to the concentration observed in beef muscle (Table 1, see also below). As far as rabbit samples are concerned, Kozioł et al. measured an a* value of 3.15 in samples 7 hours post mortem [48]. This is comparable to our measured a* value of 3.01 for samples with 0.1% rabbit myoglobin. The concentration naturally present in rabbit meat (Table 1) is comparable to what was used in our study.

Finally, the yellowness (b*) decreased to a similar extent upon addition of both myoglobins, both in raw and in sterilized products (FIG. 8B). The differences in b* values were not prominent between raw samples with and without myoglobin (23.34 and 21.71, respectively). Baking caused a slight decline in b* values, and this effect was increased in the presence of myoglobin. This suggests that adding myoglobin and baking the samples dampens the yellow tones contributed by the bulk soy matrix.

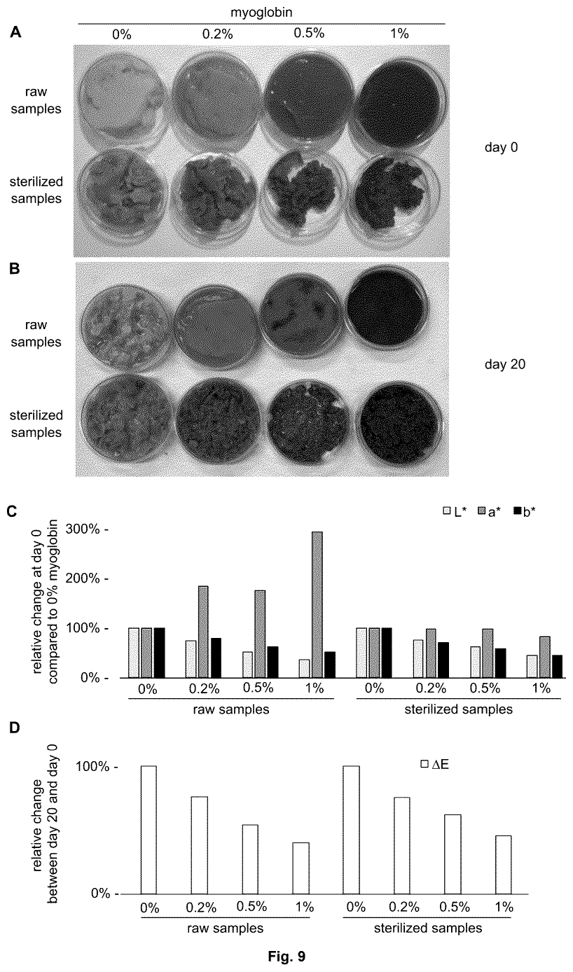

In a similar approach, we examined the color of plant-based wet pet food samples containing myoglobin obtained by precision fermentation in Pichia pastoris at different final concentrations (0, 0.2, 0.5, 1% w/w) either raw or right after heat sterilization. In raw products the lightness (L*) decreased while its redness (a*) increased with increasing myoglobin concentration (FIGS. 9A & C). Lightness also decreased with the myoglobin concentration in heat-sterilized products (FIGS. 9A & C). The yellowness (b*) decreased as myoglobin concentration increased both in raw and in sterilized products (FIGS. 9A & C). Altogether, this suggests that adding myoglobin dampens the undesirable yellow tones contributed by the plant-derived bulk protein in a product meant to mimic meat.

Besides L* a* b* values, two other factors correlated with perceptual color attributes are also often used. The first is the chroma (C*), that refers to the vividness or dullness of a color [49]. In plant-based plant-based wet pet food samples, C* values decreased (from 22.8 for raw samples without myoglobin down to 9.3 for sterilized samples with 1% myoglobin, respectively) with increasing myoglobin concentration, indicating a more vivid color. Interestingly, C* values measured in our samples with cattle myoglobin (as shown in FIG. 8) were slightly higher than those reported by Hernandez et al. [47], for beef, (24.5 and 16.93, respectively), and four times higher in samples with rabbit myoglobin than those reported by Kozioł et al. (20.1 and 5.28, respectively). This indicates that myoglobin obtained by precision fermentation might aid in achieving a more vivid product color during manufacturing.

The second factor often used in colorimetry is the hue angle (hab), which is the angle with the a* axis, that correlates with the color description in common language (red, yellow, green, blue, etc.) [49]. Meat colors are located in the first quadrant of the a*b* plane (a*, b*>0, 0≤hab≤90°), with larger values of hab indicating less red colors. Here, hab values of the samples were in the positive range (1.45-0.96, for raw samples without myoglobin and raw samples with myoglobin at day 0, respectively), and decreased with increasing myoglobin concentration. The low and decreasing hue angle values indicate a prominent redness when plant-based pet food samples contain added myoglobin. Of note, hab values measured in our samples with rabbit myoglobin were in line with those measured by Kozioł et al. in rabbit meat (1.42 and 1.07, respectively).

Finally, we measured the color difference in the plant-based wet pet food samples after 20 days of refrigerated storage, the observed color difference was reduced with increasing myoglobin concentration, to a similar extent in raw and sterilized products (FIGS. 9B & D).

Overall, these results show that the addition of myoglobin in a plant-based meat substitute either stabilizes and/or improves its desired color profile over time during cold storage, regardless of the raw or cooked state of the product. The addition of myoglobin obtained by precision fermentation increased the redness and reduced the yellowness of a plant-based wet pet food product, and hence its desirable appearance of the product. Differences were observed between myoglobin of different species, where most measured parameters corresponded to findings reported in the literature for the corresponding conventional meat product. This indicates that using myoglobin of different species, produced by precision fermentation, might contribute to a more authentic organoleptic experience when replacing conventional meat with plant-based alternatives.

### Example 6: Aromatic Analysis

Like human food, palatability plays an important role in the success of pet food products on the market. Palatability depends on the appearance, odor, taste, and texture of the product [50]. The aromatic profile of numerous food products that might be used as raw materials for pet food has been extensively studied [51-55]. Pet foods are complex objects for aromatic composition studies because of their versatile formulation. They may contain different grains (barley, oats, rice, wheat, etc.), meat sources (beef, chicken, pork, duck, turkey, venison, buffalo, etc.), animal and/or vegetable oils, as well as be supplemented with vitamins and minerals, antioxidants, and other additives [56].

Koppel et al. detected 54 kinds of volatile compounds in dry dog food, including aldehydes, esters, acids, alcohols, pyrazines, terpenes, furan, and ketones, among which aldehydes and ketones were the main volatiles [57]. Yin et al. reported a total of 55 volatile compounds in dog food sprayed with six palatability enhancers [58]. The volatile compounds were identified using headspace solid-phase microextraction (HS-SPME) coupled with gas chromatography-mass spectrometry (GC-MS). Nine compounds that were found to significantly contribute to the palatability of dog food were heptanal, nonanal, octanal, (E)-2-hexenal, (E,E)-2,4-decadienal, 2-pentylfuran, 4-methyl-5-thiazoleethanol, 2-furfurylthiol, and (E)-2-decenal. The contributions of nine key aroma compounds were further analyzed by a preference test. (E)-2-decenal, 2-furfurylthiol, and 4-methyl-5-thiazoleethanol showed higher first choice, consumption ratio, and unit contribution rate (relative amount of additional food consumed per quantity of sprayed volatile), and were vital to the overall preferred aroma of dog food.

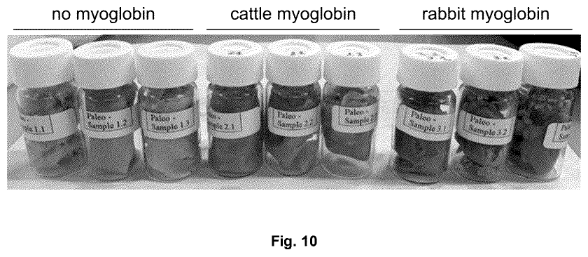

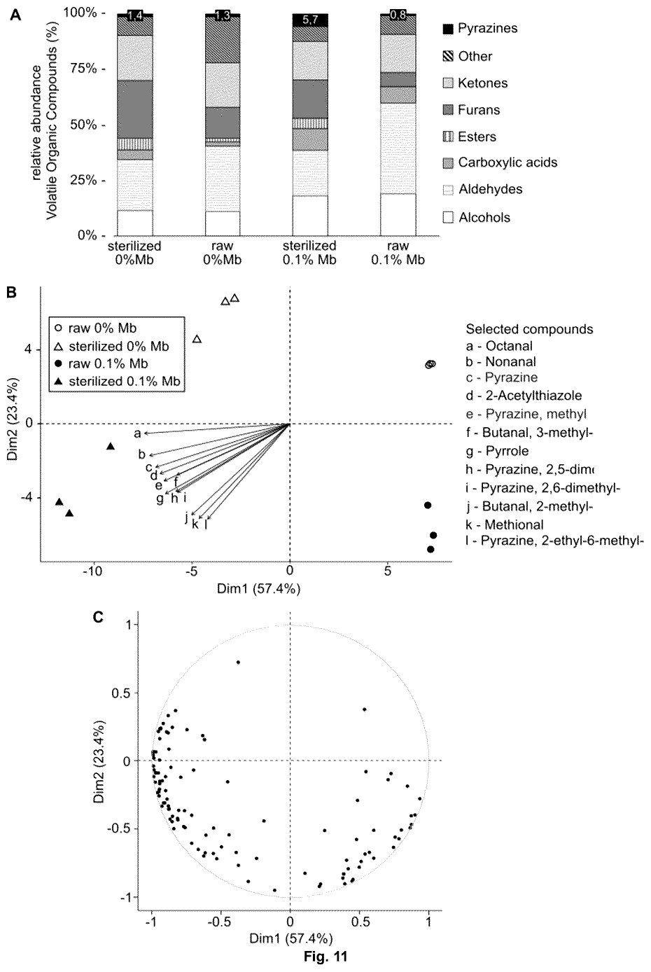

Myoglobin is not only important for meat color, but also for its sensory quality and has been associated with a serum-like taste and metallic mouthfeel of meat [59]. During meat cooking, aroma formation occurs essentially by lipid oxidation and the Maillard reaction. The latter takes place between amino groups in proteins and carbonyl groups from reduced sugars and/or reaction products of lipid oxidation. Heme iron can influence these reactions and/or their kinetics [60]. In this study, animal myoglobin was obtained by precision fermentation in Pichia pastoris and added to a model plant-based wet pet food formulation (FIG. 10). The raw and heat-sterilized samples were then subjected to headspace solid-phase microextraction (HS-SPME) followed by gas chromatography-mass spectrometry (GC-MS) in order to determine their aromatic profile.

Up to 1561 distinct aromatic compounds, depending on the heat treatment and presence of myoglobin, were detected in plant-based wet pet-food samples. Of those, between, 111 were identified from the deconvoluted peaks and used for compound class and PCA analysis (FIG. 11). For the purpose of this study, aromatic compounds that are known to convey meat-like aroma and/or are produced during the Maillard reaction were selected and further analyzed (FIG. 11, 12).

The aromatic profile of raw samples with and without 0.1% myoglobin was comprised primarily of aldehydes (29.5% and 40.7%, respectively), followed by alcohols and ketones (FIG. 11A). Heat-sterilizing the samples caused a decrease in aldehydes (23.0% and 20.7%, respectively), while the proportions of alcohols and ketones stayed relatively stable. Both the sterilized samples with and without myoglobin showed a two-fold increase in the amount of furans (6.2% and 17.2% for sterilized samples without and with myoglobin, respectively), compared to their unbaked counterparts (3.7% and 1.6% without and with myoglobin, respectively). Caramel-like, sweet, fruity, nutty, meaty, and burnt odor impressions are associated with this class of compounds [61].

A principal component analysis (PCA) was conducted to visualize the relationship between plant-based wet-pet food containing animal myoglobin obtained by precision fermentation in Pichia pastoris in terms of their volatile profile (of the 111 volatile compounds detected), with 3 repeats per sample. The PCA biplot (combining a score and loading plot) (FIG. 11B) shows that the first two principal components (PC) explained 80,8% of the total variability. A selection of volatile organic compounds (loadings, n=12) are depicted by an arrow and their compound name on the right side of the graph (FIG. 11B).

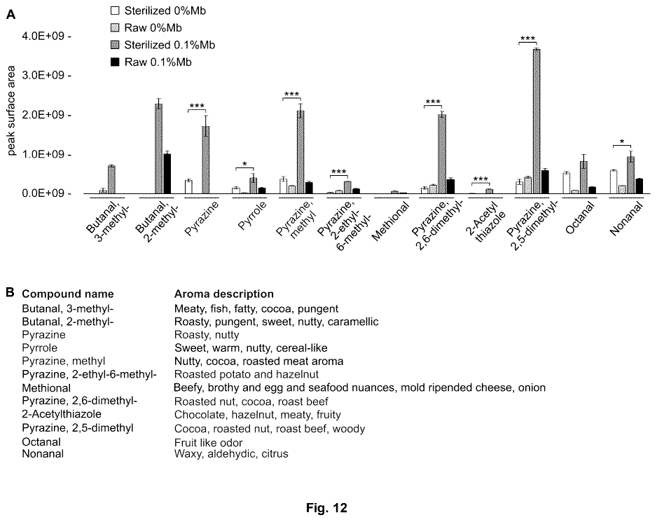

The first PC, accounting for 57.4% of variance, separates the raw from the heat sterilized plant-based wet pet food samples. Additional contribution of PC2, explaining 23.4% of variance, separates the samples with 0.1% w/w myoglobin from the samples without myoglobin. In the resulting score plot, the technical repeats (n=3) cluster together and the different samples are clearly separated. The shown loading vectors of 12 selected compounds (selected based on their importance in meat aroma-see also FIG. 12B) are strongly contributing to the sterilized 0.1% w/w myoglobin sample group (FIG. 11B). A loading plot demonstrates how each of the 111 identified volatile organic compounds influences the two main principal components (FIG. 11C). The volatile organic compounds are mainly contributing to the quadrants of the samples with 0.1% w/w myoglobin. The highest concentration of loadings is observed in the lower-left quadrant, which corresponds to the sterilized plant-based wet pet food with 0.1% of animal myoglobin obtained by precision fermentation in Pichia pastoris.

Most interestingly, the presence of myoglobin combined with a heat treatment, caused a four-fold increase in the amount of pyrazines (1.4% and 5.7% for sterilized samples without and with myoglobin, respectively). Pyrazines are heterocyclic compounds contributing to a characteristic smell and taste of food. These compounds are responsible for the “roasted-like” aroma of broiled meat or roasted coffee beans. They are mainly formed during food thermal processing of e.g. coffee, cocoa, roasted nuts and seeds, cereals and cereal products, meat products, wine, amongst others. Their formation is closely related to the Maillard reaction, which involves the reaction between the α-dicarbonyl and amino acid groups to yield an aminoketone [62].

Besides pyrazines, several aromatic compounds were formed to a greater extent when the samples contained myoglobin obtained by precision fermentation in Pichia pastoris (FIG. 11, 12) Some of these compounds are reported to be found in conventional meat, are related to the Maillard reaction and contribute to “meat-like” taste [1-4]. The peak surface area or pyrazines, such as methylpyrazine, 2-ethyl-6-methyl pyrazine, 2,6-dimethyl pyrazine and 2,5-dimethyl pyrazine largely increases when adding just 0.1% myoglobin, compared to sterilized pet food without myoglobin (FIG. 12A). The level of several aldehydes (methional, octanal and nonanal), pyrrole, some lipid oxidation products (2-methylbutanal and 3-methylbutanal) and ketone (2-acetylthiazone) are also significantly increased in heat-sterilized samples containing myoglobin, compared to sterilized samples without myoglobin (FIG. 12A).

A 2-way ANOVA was performed to assess the effect of the interaction between heat treatment and myoglobin addition on the volatiles formed. For all selected pyrazine compounds, the interaction between the heat treatment and myoglobin addition was significant (p<0.0001). This was followed by a Tukey pairwise comparison test which confirmed that the combination of heat treatment and myoglobin presence causes a significant increase in the amount of pyrazines formed.

### Example 7: Adverse Food Reactions

Adverse food reactions (AFR) in dogs and cats are a common problem that may cause cutaneous and/or gastrointestinal signs. Food allergies (categorized as immune-mediated reactions) are one type of AFR, other reactions such as lactose intolerance are classified as non-immune reactions [63]. Limited data is available on the prevalence of AFR in companion animals, but it is estimated that 1% of dogs and cats has AFR. Based on published studies, the prevalence of AFR among dogs presented to a dermatology center with cutaneous signs is estimated between 7.6% and 12% [64]. Similar data was presented by Verlinden et al: the authors estimate that 5-15% of dogs with skin or ear disorders are allergic to food and 1-10% for cats [65].

Listed in Table 4 are the most common food allergens and their prevalence causing cutaneous AFR in dogs (n=297) and cats (n=79). In addition to this list, barley, rabbit, chocolate, kidney bean and tomato were reported as food allergens in single dogs (0.3% prevalence). Egg, barley and rabbit were also reported as allergens in individual cats (1.2% prevalence) [66].

TABLE 4
Most common reported food allergens causing cutaneous
adverse food reactions in dogs (n = 297)
and cats (n = 79). Source [66].

| Food product | Dogs | Cats |
|---|---|---|
| Beef | 34% | 18% |
| Dairy products | 17% | 4% |
| Fish | — | 17% |
| Chicken | 15% | 5% |
| Wheat | 13% | 4% |
| Lamb | 5% | 3% |
| Corn | — | 4% |

Moreover, Dodds and colleagues studied the immunological reactivity of cats' saliva (n=1000), which demonstrated that low reacting foods were lamb, cow milk, pork, turkey, wheat and white-colored fish, whereas high reacting foods were millet, white potato, rice and salmon [67]. Importantly, we conducted a literature search and could not retrieve any report of myoglobin causing AFR in cats and dogs were found. These findings are also supported by a study that identified the allergens responsible for canine AFR to lamb, beef and cow's milk [68]. All the dogs (n=10) had IgE against bovine (cattle) IgG. In addition, this was the only allergen detected in cow's milk. In lamb and beef, the major allergens had a size between 51-58 kDa, which were identified as phosphoglucomutase and the IgG heavy chain. Other IgE-binding proteins with molecular masses of 27, 31, 33, 37 and 42 kDa were also detected in some sera samples. As the size of the myoglobin proteins presented in this patent is around 17 kDa, we can conclude that the authors did not detect any IgE-binding with myoglobin [68].

In addition to a literature search, we also conducted bio-informatic in silico analyses. Based on the amino acid sequence of the different myoglobin proteins (tuna (Thunnus orientalis), the steppe mammoth (Mammuthus trogontherii), the woolly mammoth (Mammuthus primigenius), rat (Rattus norvegicus), mouse (Mus musculus), rabbit (Oryctolagus cuniculus) and cattle (Bos taurus)), a sequence homology search was conducted using the AllergenOnline database version 21. A precautionary search method (80 amino acid alignment, described in the method section) was used. No significant homologies with the allergens present in the database were identified for the different myoglobin AA sequences. Significant homology was defined following the CODEX Alimentarius guidelines 2003 [69], where an identity match of more than 35% was defined as a threshold to consider the possibility of cross-reactivity.

The AllergenOnline database consists of a peer reviewed allergen list (2290 protein sequence entries of unique proven or putative allergens from 412 species). Due to the high amount of research on human allergenicity, the drawback of using this database is that most of the entries are human allergens. However, dog allergies show high similarity to human allergies (especially atopic dermatitis) [70], supporting the performed bio-informatic prediction search of the AllergenOnline database. Accordingly, showed that the identified canine beef and lamb allergens are similar to those found for meat allergy in humans.

In summary, these results indicate the different myoglobin proteins (tuna (Thunnus orientalis), the steppe mammoth (Mammuthus trogontherii), the woolly mammoth (Mammuthus primigenius), rat (Rattus norvegicus), mouse (Mus musculus), rabbit (Oryctolagus cuniculus) and cattle (Bos taurus)) are not likely to cause AFR in pets consuming these functional ingredients.

### Example 8: Materials and Methods

The following materials and methods have been used in Examples 1 to 7.

### Sequence Analysis

The sequence coding for myoglobin from Rattus norvegicus, Oryctolagus cuniculus, Mus musculus, Thunnus orientalis and Bos taurus were obtained from Uniprot (accession numbers: Q9QZ76, P02170, P04247, P68190, and P02192, respectively). The sequence coding for myoglobin from the steppe mammoth (Mammuthus trogontherii) was obtained after DNA extraction from a molar sample from the so-called Adycha specimen, Illumina DNA sequencing, merging the reads and mapping them against the African savannah elephant (Loxodonta africana) genome [23]. Multiple sequence alignments were performed with Clustal Omega and visualized using Jalview 2.11.1.4 [72].

### Protein Modelling

The structure of the 6 myoglobins listed above (see 3.1) was modelled with the automated protein structure homology-modelling server SWISS-MODEL (Waterhouse et al. 2018). For the 5 mammalian species, the high resolution X-raystructure of the deoxymyoglobin from Sus scrofa (RCSB Protein Data Bank (PDB) accession number: 1myg.1.A) was used as template. For the myoglobin from the Pacific bluefin tuna, the X-ray structure from the blackfin tuna myoglobin (PDB accession number 2nx0.1) was used as template. All protein structures were visualized and figures generated using DeepView v4.1 [73].

### Construction of Expression Plasmids

Myoglobin coding sequences were codon optimized for expression in Pichia pastoris (Komagataella phaffii) [74]. The optimized sequences encoding the myoglobin from Bos taurus and Mammuthus trogontherii, preceded by the coding sequence of the Saccharomyces cerevisiae mating factor alpha were chemically synthetized by GenScript. Gene fragments were cloned into the pBDIPp5 vector (Bos taurus and Mammuthus trogontherii), downstream of the AOX1 promoter and upstream of the AOX1 terminator, the HIS4 selection marker and the AOX1 3′ fragment. For the other four myoglobins, the optimized sequences preceded by the coding sequence of the Saccharomyces cerevisiae mating factor alpha and the AOX1 promoter, and followed by the AOX1 terminator and the HIS4 selection marker, were chemically synthesized by IDT. Gene fragments were cloned into the pFL38 vector, so as to replace the original URA3 selection marker. Vectors were amplified in E. coli (DH10B), purified with the SmartPure Plasmid Kit (Eurogenetec) and verified by Sanger sequencing (Eurofins Genomics). Expression cassettes were generated from the resulting vectors by restriction of the plasmids with BgIII (New England Biolabs), which cuts upstream from the AOX1 promoter and downstream from the AOX1 3′ fragment.

### Strains Engineering

The Pichia pastoris (Komagataella phaffii) strain GS115 (his4) was obtained from Life Technologies. Cell transformation was performed using the electroporation method essentially as previously described [75]. Briefly, cells of the GS115 strain were grown in YPD (1% yeast extract, 2% peptone and 2% D-glucose) medium. Cells in the exponentially growing phase were incubated for 30 min in YPD medium with 200 mM HEPES buffer (pH 8.0) and 25 mM dithiothreitol. Competent cells were then washed with ice-cold 1M sorbitol, and transferred to a sterile electroporation cuvette (Bio-Rad). Cells were then electroporated with 1-5 μg of linear expression cassette (see 3.3) using a Gene-Pulser (Bio-Rad) electroporator, and resuspended in 1 mL of YPD medium containing 1M sorbitol before transfer to a sterile 1.5-mL eppendorf tube. Cells were incubated at 28° C. without agitation for 3 h, before plating onto agar plates containing solid MGY medium (Minimal Glycerol Medium: 1.34% Yeast Nitrogen Base with ammonium sulfate without amino acids, 2% D-glucose, 4 10-5% biotin with 2% agar). Plates were incubated for up to 4 days at 28° C. Transformants able to grow in the absence of histidine were verified by PCR. A DNA fragment specific for the myoglobin expression cassette was amplified using a pair of primers that anneal with the coding sequence of the Saccharomyces cerevisiae mating factor alpha. PCR reactions were conducted using the OneTaq® Quick-Load® 2X Master Mix with Standard Buffer (New England Biolabs) according to the manufacturer's instructions. PCR products were visualized after migration at 60V for 60 min in a 2% agarose TAE gel with ethidium bromide. The Quick-Load® Purple 1 kb Plus DNA Ladder (New England Biolabs) was used to control the size of the amplified fragment.

Positive clones were then screened for their ability to express Mb. Cells were grown in deep, 96 wells microtiter plates in BMGY medium (1% yeast extract, 2% peptone, 100 mm Potassium Phosphate buffer (pH6), 1.34% Yeast Nitrogen Base with ammonium sulfate without amino acids, 4 10-5% biotin, 1% glycerol). 1% methanol was added to exponentially growing cells, to induce Mb expression. Samples were collected 72 h after the start of the methanol induction, and analyzed by SDS-PAGE to assess Mb levels (see below).

The best producing strains were selected for further experiments, and were cryopreserved as a master cell bank at −80° C. prior to use. Results shown here were obtained using the PAL004 (cattle myoglobin) and PAL096 (rabbit myoglobin) strains.

### Recombinant Protein Production and Purification

Cells from the master cell bank were plated on a YPD agar (1% yeast extract, 2% soy peptone and 3% D-glucose, 2% agar) plate and incubated for 2 days at 28° C. A seed culture was then prepared in a 2 L flask containing 200 ml of YPG (1% yeast extract, 2% soy peptone and 3% glycerol) medium, and incubated for 24 h at 28° C. in an orbital shaker incubator. This seed culture was then used to inoculate a glass-vessel fermenter (Biostat B, Sartorius) containing 900 ml of modified BSM medium [76]. A batch fermentation phase was conducted for about 14-16 h, until all the glycerol was consumed. A glycerol fed batch phase was then conducted to further increase the biomass. The temperature was then lowered to 26° C., and a mixed glycerol: methanol was applied for 72 h to induce Mb expression. Dissolved oxygen was maintained above 25% during the whole fermentation, while the pH was kept at 6 during the growth phase and 5 during the induction phase. After 96 h of methanol induction, cells were removed by centrifugation at 4,000 rpm at 4° C. Remaining cells were removed by microfiltration using cellulose filters with a pore diameter of 0.45 μm (Sartorius Hydrosart) using a tangential flow filtration device (Sartoflow, Sartorius). After ultrafiltration using cellulose filters with a molecular weight cut-off of 10 kD using the same device, the product was stored frozen at −20° C. until use. When necessary for the assay, the ultrafiltration retentate was further concentrated using disposable ultrafiltration centrifuge devices, with a polyethersulfone membrane having a molecular-weight cutoff of 10 kD (Pierce Protein Concentrators, Thermo Scientific).

### Sodium Dodecyl Sulfate Polyacrylamide Gel Electrophoresis (SDS-PAGE)

For protein electrophoresis, 20 μl of cell-free supernatant were mixed with an equal volume of 2X protein loading buffer (100 mM Tris (pH6,8); 4 mM EDTA, 4% SDS, 20% glycerol; 0.02% bromophenol blue, 4% β-mercaptoethanol). Samples were incubated at 95° C. for 5 minutes, and the appropriate volume loaded in a 15% polyacrylamide gel on a vertical mini-PROTEAN gel apparatus (Bio-Rad) at 200 V for 40 minutes. Myoglobin from horse heart muscle (Sigma) was used as standard. The molecular weight marker (PageRuler™ Prestained Protein Ladder) was purchased from Thermofisher. After electrophoresis, proteins in the gel were stained with Coomassie Brilliant Blue G-250 (Bio-Rad).

### Plant-Based Meat Substitutes as Pet Food

The base formulation of plant-based wet pet food comprised out of 20% w/w soy protein isolate (Bodyfit, NL), 10% w/w canola oil and 70% w/w tap water. To reach a 0.1% w/w level of myoglobin in the pet food, a certain volume of tap water was replaced with myoglobin solution. This depended on the concentration of myoglobin in the solution. The matrix was mixed for 3 minutes with a handheld mixer. 10 g of the matrix was transferred with a plastic syringe to a 250 mL Schott bottle. Baking was performed either at 121° C. for 30 minutes in a pressure cooker (for aromatic analyses).

### Colorimetry Analyses

First, color stability during time was measured in selected cattle myoglobin samples. These were initially frozen at −20° C. immediately after production and kept frozen and in the dark until use. At the start of the experiment, samples were wrapped in aluminium foil to keep from light and placed to thaw at 4° C. for 4 hours. Samples were kept in these conditions for 2 months and sampled in non-equidistant intervals.

Prior to colorimetric measurements, dilutions were made with distilled water so that a total of 10 mg was present in 30 mL of sample for colorimetry, allowing for reproducible measurement of color values. Prior to performing sample color readings, the colorimeter (HunterLab ColorFlex EZ Model 45/0, Hunter Associates Laboratory Inc., Rustin, VA, USA) was calibrated using standard black and white tiles. Color readings (where L* describes the lightness of the sample (0=black, 100=white), a* describes ranges from green (−) to red (+), and b* describes ranges from blue (−) to yellow (+)) were recorded with a D65 illuminant and a 10° standard observer using the Commission Internationale de l'Eclairage (CIE L*a*b*) color scale. The same volume of each sample was then placed into a glass cuvette and read for L*a*b* values. EasyMatch QC version 4.98 software (Hunter Associates Laboratory Inc., Rustin, VA, USA) was used for data capturing. Color difference (ΔE) values were calculated according to the following equation, where L, a, b are the values of the sample, and L₀, a₀, and b₀ are the initial color values:

$$Δ E = \sqrt{{( L - {L}_{0} )}^{2} + {( a - {a}_{0} )}^{2} + {( b - {b}_{0} )}^{2}}$$

Second, color values were read in plant-based wet pet food with 0, 0.1, 0.2, 0.5 and 1% w/w cattle or rabbit myoglobin added. The plant-based wet pet food comprised of 20% w/w soy protein isolate (BodyFit NL), 10% w/w canola oil and 70% w/w of H₂O. Varying volumes of myoglobin solution were added to this mixture, replacing the volume of water, to get matrices with the desired myoglobin concentration. One part of the samples was kept raw and the other was placed into at 250 ml Schott bottle and thermically processed by autoclaving for 30 mins at 121° C. All the samples were then transferred to 35 mm plastic Petri dishes. Readings were carried out in these dishes as they can fit in the measuring crevice of the colorimeter. The measuring procedure is described above. In some cases, the samples were placed at 4° C. and left covered for 20 days, after which a colorimetry measurement was performed. Chroma (C*) and the hue angle (hab) were calculated using the following formulas [49]:

$$C * = \sqrt{{a}^{* 2} + {b}^{* 2}}$$

$${h}_{a b} = arctan \frac{{b}^{*}}{{a}^{*}}$$

### Aromatic Analyses

70 g of model plant-based wet pet food was prepared as described in Paragraph 3.8. To this base matrix, 0.1% w/w of cattle myoglobin was added. The samples were thermically processed at 121° C. for 30 minutes, and let to cooled down to room temperature. 2 g of pet food samples were then transferred to 20 mL glass vials for aromatic profile analysis.

Aromas from plant-based meat substitutes containing no myoglobin or myoglobin were analyzed using solid phase microextraction (SPME) in combination with gas chromatography coupled to mass spectrometry (GC-MS). All samples were tested raw (before baking) and after baking for 30 minutes at 121° C. in a pressure cooker. Samples were analyzed in triplicates and in batches, to minimize the time spent at room temperature. For each sample, 2 g of material were inserted in a 20 mL for extraction using a PDMS/DVB/CAR (grey) SPME fiber, an incubation temperature of 45° C. for 20 min, and an extraction time of 40 minutes. Extracted compounds were injected in a DB-5 MS (30 m×0.25 mm×1 μm) column, using a splitless injector at 250° C. TOF detection was performed in scan mode (35-350 amu, 10 scans/s). SPME-GC-MS profiles were processed for peak deconvolution and compound identification.

### Texture Analyses

For texture profile analyses, 500 g (with 0.1% cattle myoglobin) or 550 g (without myoglobin) the base formulation of model wet pet food was made, as described in Paragraph 3.8. A silicon tray with cube shaped molds (3.3×3.3×3.3 cm) was filled using a syringe with 37 g (40 mL) of the model wet pet food, and cooked at 180° C. for 15 minutes in an oven with a full tray of water (creating a steam oven). 12 replicates of cubes with and without myoglobin were made. Samples were then wrapped in aluminium foil and stored in containers at 4° C. overnight, after which analysis was performed.

To obtain measurements for the firmness (defined as peak force [g]) and adhesiveness (defined as negative peak force [g]), a TPA was performed. The TPA sequence involved contacting, compressing, and withdrawing from the product to the original contact point, then repeating the entire cycle a second time. Lloyd LF Plus Texture Analyzer with accompanying computer software was used to measure the force-time curve. Samples were compressed under a 5 cm diameter cylinder probe at a test speed of 2 mm/see and a compression distance of 1 cm for 5 sec. Differences in the different texture parameters between samples with or without myoglobin were evaluated by student t-test.

### Adverse Reactions Prediction

Allergenicity was assessed by running a homology search of the myoglobin amino acid protein sequences listed above on the AllergenOnline database, i.e. a comprehensive list of putative allergenic proteins developed for the purpose of food safety evaluation. An 80-mer sliding window homology search was carried out with default parameters. Any sliding window with a sequence showing an identity greater than 35% to any of the sequences stored in the database would be considered a match and hence indicate a possible cross-reactivity with the known allergen.

In addition to in silico allergenicity prediction, a literature search was performed on the Google Scholar and Pubmed databases, using the following search queries: “myoglobin allergy cat”, “myoglobin allergy dog”, “myoglobin allergy pet”.

### Example 9: Additional Dog Food Product Comprising Isolated Rabbit Myoglobin

Production of Purified Rabbit Myoglobin by Escherichia coli.

Full length rabbit myoglobin gene SEQ ID NO: 2 is synthesized and cloned in a modified pET-23a (+) vector, comprising the T7 promoter and terminator and a C-terminal hexa His-tag (Genscript Biotech, Leiden, the Netherlands). The ampR marker gene originally present in said vector is replaced by the proBA operon from E. coli strain K12, including its original transcription regulatory elements, to facilitate selection without antibiotics. The correctly assembled plasmids are confirmed by PCR and used to transform a proline auxotrophic E. coli protein production strain (E. coli K12 ΔproBA). Transformed strains are incubated overnight in shake-flasks containing minimal medium (10.5 g/L K₂HPO₄, 4.5 g/L KH₂PO₄, 1.0 g/L (NH₄)₂SO₄, 0.12 g/L MgSO₄, 0.5 g/l Nacitrate, 2 g/L glucose, and 5.0 mg/L thiamine.HClat 37° C. and 150 rpm (pH 6). 500 μl of the overnight culture is transferred to a 1 L shake-flask containing 500 mL minimal medium and incubated at 37° C. and 150 rpm, until an OD600 of 0.4-1 is reached. IPTG (isopropyl-β-D-thiogalactoside) at a concentration of 100 μM is added to the culture, after which the culture is incubated for 24 h at 16° C. and 150 rpm. The culture is harvested and centrifuged at 3500×g (4° C.) for 15 min. The supernatant is discarded and the pellet is dissolved in 50 mL BugBuster Protein Extraction Reagent (Novagen), containing 1 KU Lysozyme/ml (Sigma-Aldrich), 25 U Benzonase® Nuclease and complete™, EDTA-free Protease Inhibitor Cocktail (Roche). The dissolved pellet is incubated for 30 min at 4° C. in a shaker. The centrifugation step is repeated and the cell-free extract (supernatant) is collected and assayed by SDS-PAGE to confirm the production of myoglobins. The production of rabbit myoglobins by the respective transformed strains is confirmed by the presence of protein bands of the correct size.

For purification, the cell-free extracts containing the soluble fraction of proteins is loaded to a HisTrap FF 1 mL column (Cytiva, MA, USA), coupled with an AKTA start system. The column is equilibrated with 20 mM HEPES, 0.4 M NaCl, and 20 mM imidazole, pH 7.5, 1 mL/min flow rate. The protein is eluted with 20 mM HEPES, 0.4 M NaCl, and 400 mM imidazole, pH 7.5. The fractions containing the rabbit myoglobins are pooled, concentrated and confirmed by SDS-PAGE and Western Blotting using an anti-histidine tag antibody (Bio-Rad), which confirm the successful purification of all myoglobins. The purified rabbit myoglobins are stored at −20° C. for later use.

### Production of Pet Food

100 kg of dry dog food control product was prepared with ingredients in the proportions shown in table 5. The ingredients for the dry basal were weighed and placed into mixer. These were blended and milled with a hammer-mill and conveyed to a holding bin.

TABLE 5
dog food recipe

| Ingredient | kg |
|---|---|
| Whole cereal grains | 430 |
| Vegetable | 35 |
| Egg | 8 |
| Minerals premix | 11 |
| Vitamina premix | 5.5 |
| Potassium Sorbate | 4 |
| Fish oil | 7 |
| Isolated rabbit myoglobin | 70 |

The dry basal mix was fed to an extruder at 250 kg/hr and the fish oil at 2.4 kg/hr. Water (10 L/hr) and steam (27 L/hr) was added to the ingredient mix in the extruder barrel. The extruder screw speed was 900 rpm and conditions of the molten mass maintained at 20 bars and at least 104° C. As the ropes of expanded mass exited the extruder dies, they were cut into kibbles by knives rotating at 900 rpm across the face of the dies. The kibbles were fed into a drier and dried at 125° C. for 21 minutes to a moisture content of 9.2%. The coated kibbles were tumbled for an additional 5 minutes after all the liquids were sprayed.

A small amount of the kibbles are sampled for color stability, aromatic and nutritional analysis. The coated kibbles were then filled into poly bags and sealed.

### Example 10: Additional Cat Food Product Comprising Isolated Rat Myoglobin

1 kg of homemade cat food product was prepared with ingredients in the proportions shown in Table 6. The ingredients were weighed and placed into a mixer. These were blended with a blender. After the mix is blended, isolated myoglobin is added to the mix, and blended for another 2 mins. The isolated myoglobin in power shape is measured with a home balance. Alternatively, if the isolate myoglobin is in cube shape, each cube is 10 g, then 5 cubes are added to the mix, followed by additional 2 mins of blending. The mix is then placed in a mould, and baked for 30 min at 180° C. in an oven. The mix may also be further processed into a drier to a moisture content of about 10%.

TABLE 6
Home made cat food recipe

| Ingredient | g |
|---|---|
| Whole cereal grains | ~700 |
| Vegetable | ~250 |
| Egg | 50 |
| Minerals premix | 10 |
| Vitamina premix | 5 |
| Fish oil | 5 |
| isolated rat myoglobin | 50 |

It is possible to modify the ingredients, and alternative types of ingredients may be added. The vegetable may include spinach, lettuce, kale, lentils, peas, chickpeas, or beans. The cereals may include rice, corn, barley, oats, wheat. Tubers may include potatoes, sweet potatoes. Sprouts may include soy, lentils, rye, sesame. Flowers may include artichokes, broccoli, cauliflower. Stems may include celery, asparagus. Seeds may include pumpkin, sunflower.

### REFERENCES

ADDIN Mendeley Bibliography CSL_BIBLIOGRAPHY 1. Migita, K.; liduka, T.; Tsukamoto, K.; Sugiura, S.; Tanaka, G.; Sakamaki, G.; Yamamoto, Y.; Takeshige, Y.; Miyazawa, T.; Kojima, A.; et al. Retort Beef Aroma That Gives Preferable Properties to Canned Beef Products and Its Aroma Components. Anim. Sci. J. 2017, 88, 2050-2056, doi: 10.1111/ASJ.12876.
2. Garcíα-lomillo, J.; González-sanjosé, M. L.; Pino-garcía, R. Del; Ortega-heras, M.; Muñiz-rodríguez, P. Effect of a New Natural Seasoning on the Formation of Pyrazines in Barbecued Beef Patties. 2016, 2016.
3. Watanabe, A.; Kamada, G.; Imanari, M.; Shiba, N.; Yonai, M.; Muramoto, T. Effect of Aging on Volatile Compounds in Cooked Beef. Meat Sci. 2015, 107, 12-19, doi: 10.1016/J.MEATSCI.2015.04.004.
4. Vilar, E. G.; O'Sullivan, M. G.; Kerry, J. P.; Kilcawley, K. N. Volatile Organic Compounds in Beef and Pork by Gas Chromatography-Mass Spectrometry: A Review. Sep. Sci. Plus 2022, 5, 482-512, doi: 10.1002/SSCP.202200033.
5. Brantley, R. E.; Smerdon, S. J.; Wilkinson, A. J.; Singleton, E. W.; Olson, J. S. The Mechanism of Autooxidation of Myoglobin. J. Biol. Chem. 1993, 268, 6995-7010, doi: 10.1016/s0021-9258 (18) 53138-0.
6. Fleming, H. P.; Blumer, T. N.; Craig, H. B. Quantitative Estimations of Myoglobin and Hemoglobin in Beef Muscle Extracts. J. Anim. Sci. 1960, 19, 1164-1171, doi: 10.2527/jas1960.1941164x.
7. Rickansrud, D. A.; Henrickson, R. L. Total Pigments and Myoglobin Concentration in Four Bovine Muscles. J. Food Sci. 1967, 32, 57-61, doi: 10.1111/j. 1365-2621.1967.tb01957.x.
8. Seideman, S. C.; Cross, H. R.; Smith, G. C.; Durland, P. R. Factors Associated with Fresh Meat Color: A Review. J. Food Qual. 1984, 6, 211-237, doi: https://doi.org/10.1111/j.1745-4557.1984.tb00826.x.
9. Texas, A. Meat Color-Meat Science Available online: https://meat.tamu.edu/ansc-307-honors/meat-color/(accessed on 6 Sep. 2023).
10. Brown, D. W. The Concentration of Myoglobin and Hemoglobin in Tuna Flesh. J. Food Sci. 1962, 27, 26-28, doi: https://doi.org/10.1111/j.1365-2621.1962.tb00052.x.
11. Bu, Y.; Han, M.; Tan, G.; Zhu, W.; Li, X.; Li, J. Changes in Quality Characteristics of Southern Bluefin Tuna (Thunnus Maccoyii) during Refrigerated Storage and Their Correlation with Color Stability. Lwt 2022, 154, 112715, doi: 10.1016/j.lwt.2021.112715.
12. Sánchez-Zapata, E.; Amensour, M.; Oliver, R.; Fuentes-Zaragoza, E.; Navarro, C.; Fernández-López, J.; Sendra, E.; Sayas, E.; Pérez-Alvarez, J. A. Quality Characteristics of Dark Muscle from Yellowfin Tuna Thunnus Albacares to Its Potential Application in the Food Industry. Food Nutr. Sci. 2011, 02, 22-30, doi: 10.4236/fns.2011.21003.
13. Ochiai, Y.; Nurilmala, M. Methods for Evaluating the Quality of Tuna Meat. J. Ocean Technol. 1997, 33, 24-29.
14. Giovane, A.; Greco, G.; Maresca, A.; Tota, B. Myoglobin in the Heart Ventricle of Tuna and Other Fishes. Experientia 1980, 36, 219-220, doi: 10.1007/BF01953742.
15. Cross, H. R.; Singh, R. P. Meat Processing-Protein, Fatty Acid, and Nutrient Content | Britannica Available online: https://www.britannica.com/technology/meat-processing/Protein (accessed on 6 Sep. 2023).
16. Ouhayoun, J.; Dalle Zotte, A. Muscular Energy Metabolism and Related Traits in Rabbit. A Review. World Rabbit Sci. 1993, 1, 97-108.
17. Noren, S. R.; Williams, T. M. Body Size and Skeletal Muscle Myoglobin of Cetaceans: Adaptations for Maximizing Dive Duration. Comp. Biochem. Physiol.-A Mol. Integr. Physiol. 2000, 126, 181-191, doi: 10.1016/S1095-6433 (00) 00182-3.
18. Castellini, M. A.; Somero, G. N. Buffering Capacity of Vertebrate Muscle: Correlations with Potentials for Anaerobic Function. J. Comp. Physiol.=B 1981, 143, 191-198, doi: 10.1007/BF00797698.
19. Ismaeel, A.; Laudato, J. A.; Fletcher, E.; Papoutsi, E.; Tice, A.; Hwa, L. S.; Miserlis, D.; Jamurtas, A. Z.; Steiner, J.; Koutakis, P. High-Fat Diet Augments the Effect of Alcohol on Skeletal Muscle Mitochondrial Dysfunction in Mice. Nutrients 2022, 14, 1-14, doi: 10.3390/nu14051016.
20. Åkeson, Å.; Ehrenstein, G. v.; Hevesy, G.; Theorell, H. Life Span of Myoglobin. Arch. Biochem. Biophys. 1960, 91, 310-318, doi: 10.1016/0003-9861 (60) 90506-3.
21. Lawrie, R.A. Biochemical Differences between Red and White Muscle. Nature 1952, 170, 122-123, doi: https://doi.org/10.1038/170122a0.
22. Wajcman, H.; Kiger, L.; Marden, M.C. Structure and Function Evolution in the Superfamily of Globins. Comptes Rendus-Biol. 2009, 332, 273-282, doi: 10.1016/j.crvi.2008.07.026.
23. van der Valk, T.; Pečnerová, P.; Díez-del-Molino, D.; Bergström, A.; Oppenheimer, J.; Hartmann, S.; Xenikoudakis, G.; Thomas, J.A.; Dehasque, M.; Sağlcan, E.; et al. Million-Year-Old DNA Sheds Light on the Genomic History of Mammoths. Nature 2021, 591, 265-269, doi: 10.1038/s41586-021-03224-9.
24. Sanctorum, H.; de Jong, A. MEAT SUBSTITUTE COMPRISING ANIMAL MYOGLOBIN 2021.
25. Mirceta, S.; Signore, A. V.; Burns, J. M.; Cossins, A. R.; Campbell, K. L.; Berenbrink, M. Evolution of Mammalian Diving Capacity Traced by Myoglobin Net Surface Charge. Science (80-.). 2013, 340, 1-53, doi: 10.1126/science.1234192.
26. Isogai, Y.; Imamura, H.; Nakae, S.; Sumi, T.; Takahashi, K. ichi; Nakagawa, T.; Tsuneshige, A.; Shirai, T. Tracing Whale Myoglobin Evolution by Resurrecting Ancient Proteins. Sci. Rep. 2018, 8, 1-14, doi: 10.1038/s41598-018-34984-6.
27. Lahtinen, M.; Clinnick, D.; Mannermaa, K.; Salonen, J. S.; Viranta, S. Excess Protein Enabled Dog Domestication during Severe Ice Age Winters. Sci. Rep. 2021, 11, 7, doi: 10.1038/s41598-020-78214-4.
28. Driscoll, C. A.; Clutton-Brock, J.; Kitchener, A.C.; O'Brien, S. J. The Taming of the Cat. Sci. Am. 2009, 300, 68-75, doi: 10.4324/9780080939544-12.
29. (Hill's), C. O. Cats & Tuna: Is It Safe for Them? Available online: https://www.hillspet.com/cat-care/nutrition-feeding/can-cats-eat-tuna?lightboxfired=true #(accessed on 8 Jun. 2023).
30. Koutsoumanis, Konstantinos; Allende, Ana; Alvarez-Ordonez, Avelino; Bolton, Declan; Bover-Cid, Sara; Chemaly, Marianne; Davies, Robert; De Cesare, Alessandra; Hilbert, Friederike; Lindqvist, Roland; Nauta, Maarten; Peixe, Luisa; Ru, Giuseppe; Simmons, Mar, L. Updated List of QPS-Recommended Biological Agents for Safety Risk Assessments Carried out by EFSA. 2022, doi: 10.5281/zenodo.6902983.
31. Bill Kaelle, G.C.; Menezes Souza, C. M.; Bastos, T. S.; Vasconcellos, R. S.; Oliveira, S. G. de; Felix, A. P. Diet Digestibility and Palatability and Intestinal Fermentative Products in Dogs Fed Yeast Extract. Ital. J. Anim. Sci. 2022, 21, 802-810, doi: 10.1080/1828051X.2022.2054733.
32. De Oliveira, R. T.; Haese, D.; Kill, J. L.; Lima, A.; Malini, P. V.; Thompson, G. R. Palatability of Cat Food with Sodium Pyrophosphate and Yeast Extract. Cienc. Rural 2016, 46, 2202-2205, doi: 10.1590/0103-8478cr20151651.
33. Knight, A.; Satchell, L. Vegan versus Meat-Based Pet Foods: Owner Reported Palatability Behaviours and Implications for Canine and Feline Welfare. PLOS One 2021, 16, 1-25, doi: 10.1371/journal.pone.0253292.
34 Mao, M.; Beckmann, C. H.; Pannevis, M.; Olivares, A.; Schuetz, N. Process of Preparing a Wet Pet Food, Wet Pet Food Produced by the Process and Uses Thereof 2022.
35. The European Pet Food Industry Federation (FEDIAF) Nutritional Guidelines For Complete and Complementary Pet Food for Cats and Dogs; 2020;
36. Purina Purina ONE Real Beef & Bison Grain-Free Wet Dog Food | Purina Available online: https://www.purina.com/dogs/shop/purina-one-true-instinct-grain-free-beef-bison-wet-dog-food (accessed on 8 Jun. 2023).
37. Armstrong P. J., G. K. L., B. I . . . . D. J. Introduction to Feeding Normal Cats. In Small Animal Clinical Nutrition; Hand M. S., T. C. D., R. R. L., R. P . . . . N. B. J., Ed.; 2010; pp. 371-372.
38. Kiemer, L. Vegan Diet and Its Effects on the Dog's Health; Lithuanian University of Health Sciences, Kaunas, Lithuania, 2020;
39. Heath, A.-L. M.; Fairweather-Tait, S. J. Clinical Implications of Changes in the Modern Diet: Iron Intake, Absorption and Status. Best Pract. Res. Clin. Haematol. 2002, 15, 225-241, doi: 10.1053/beha.2002.0208.
40. Food, E. P. on F. A. and N. S. added to Scientific Opinion on the Safety of Heme Iron (Blood Peptonates) for the Proposed Uses as a Source of Iron Added for Nutritional Purposes to Foods for the General Population, Including Food Supplements. EFSA J. 2010, 8, 1-31, doi: 10.2903/j.efsa.2010.1585.
41. Hunt, M. C. Meat Color Measurements of Myoglobin. Am. Meat Sci. Assoc. Champaign, Illinois USA 2012, 33, 43.
42. Cairone, F.; Carradori, S.; Locatelli, M.; Casadei, M. A.; Cesa, S. Reflectance Colorimetry: A Mirror for Food Quality-a Mini Review. Eur. Food Res. Technol. 2020, 246, 259-272, doi: 10.1007/S00217-019-03345-6/METRICS.
43. McClements, D. J.; Grossmann, L. A Brief Review of the Science behind the Design of Healthy and Sustainable Plant-Based Foods. npj Sci. Food 2021 51 2021, 5, 1-10, doi: 10.1038/s41538-021-00099-y.
44. Bohrer, B. M. An Investigation of the Formulation and Nutritional Composition of Modern Meat Analogue Products. Food Sci. Hum. Wellness 2019, 8, 320-329, doi: 10.1016/J.FSHW.2019.11.006.
45. Muizzuddin, N. M. K., M. D. S. W., Use of a Chromameter in Assessing the Efficacy of Anti-Irritants and Tanning Accelerators. J. Soc. Cosmet. Chem 1990, 369-378.
46. McClements, D. J.; Weiss, J.; Kinchla, A. J.; Nolden, A. A.; Grossmann, L. Methods for Testing the Quality Attributes of Plant-Based Foods: Meat- and Processed-Meat Analogs. Foods 2021, 10.
47. Hernández, B.; Sáenz, C.; Alberdi, C.; Diñeiro, J. M. CIELAB Color Coordinates versus Relative Proportions of Myoglobin Redox Forms in the Description of Fresh Meat Appearance. J. Food Sci. Technol. 2016, 53, 4159, doi: 10.1007/S13197-016-2394-6.
48. Kozioł, K.; Maj, D.; Bieniek, J. Changes in the Color and PH of Rabbit Meat in the Aging Process. Med. Weter. 2015, 71, 104-108.
49. Wyszecki, G.; Stiles, W. S. Color Science: Concepts and Methods, Quantitative Data and Formulae, 2nd Edition; 2nd ed.; Wiley, 2000; ISBN 978-O-471-39918-6.
50. Boudreau, J. C.; White, T. D. Dog Food Flavors. 1978.
51. Brunton, N. P.; Cronin, D. A.; Monahan, F. J. Volatile Components Associated with Freshly Cooked and Oxidized Off-Flavours in Turkey Breast Meat. Flavour Fragr. J. 2002, 17, 327-334, doi: 10.1002/FFJ.1087.
52. Lammers, M.; Dietze, K.; Ternes, W. HEADSPACE VOLATILES OF DRY-CURED HAM: A COMPARISON OF DIFFERENT MANUFACTURING STYLES BY SPME AND GC/MS ANALYSIS. J. Food Process. Preserv. 2011, 35, 850-860, doi: 10.1111/J.1745-4549.2011.00538.X.
53. Parker, J. K.; Hassell, G. M. E.; Mottram, D. S.; Guy, R. C. E. Sensory and Instrumental Analyses of Volatiles Generated during the Extrusion Cooking of Oat Flours. J. Agric. Food Chem. 2000, 48, 3497-3506, doi: 10.1021/JF991302R.
54. Zeng, Z.; Zhang, H.; Zhang, T.; Tamogami, S.; Chen, J. Y. Analysis of Flavor Volatiles of Glutinous Rice during Cooking by Combined Gas Chromatography-Mass Spectrometry with Modified Headspace Solid-Phase Microextraction Method. J. Food Compos. Anal. 2009, 22, 347-353, doi: 10.1016/J.JFCA.2008.11.020.
55. Koppel, K.; Adhikari, K.; Di Donfrancesco, B. Volatile Compounds in Dry Dog Foods and Their Influence on Sensory Aromatic Profile. Molecules 2013, 18, 2646, doi: 10.3390/MOLECULES18032646.
56. Willard, T. Choosing and Sourcing the Best Ingredients. In Petfood Technology; Kvamme, J. L., P. T . . . , Ed.; Watt Publishing Co.: Mt. Morris, IL, USA, 2003; pp. 76-81.
57. Koppel, K.; Gibson, M.; Alavi, S.; Aldrich, G. The Effects of Cooking Process and Meat Inclusion on Pet Food Flavor and Texture Characteristics. Anim. 2014, Vol. 4, Pages 254-271 2014, 4, 254-271, doi: 10.3390/ANI4020254.
58. Yin, M.; Shao, S.; Zhou, Z.; Chen, M.; Zhong, F.; Li, Y. Characterization of the Key Aroma Compounds in Dog Foods by Gas Chromatography-Mass Spectrometry, Acceptance Test, and Preference Test. J. Agric. Food Chem. 2020, 68, 9195-9204, doi: 10.1021/ACS.JAFC.0C03088/ASSET/IMAGES/MEDIUM/JF0C03088_0005.GIF.
59. Office of Food Additive Safety GRAS Notice No. 1001 Bovine Myoglobin Produced by Pichia Pastoris; 2021; Vol. 6; ISBN 1111111111.
60. Ba, H. Van; Hwang, I.; Jeong, D.; Touseef, A. Principle of Meat Aroma Flavors and Future Prospect. Latest Res. into Qual. Control 2012, 2, 145-176.
61. Wailzer, B.; Klocker, J.; Wolschann, P.; Buchbauer, G. Structural Features for Furan-Derived Fruity and Meaty Aroma Impressions. Nat. Prod. Commun. 2016, 11, 1475-1479, doi: 10.1177/1934578X1601101014.
62. Zhao, C.; Cao, H.; Xiao, J. Pyrazines in Food. Handb. Diet. Phytochem. 2020, 1-25, doi: 10.1007/978-981-13-1745-3_44-1.
63. Tiffany, S.; Parr, J.M.; Templeman, J.; Shoveller, A.K.; Manjos, R.; Yu, A.; Verbrugghe, A. Assessment of Dog Owners' Knowledge Relating to the Diagnosis and Treatment of Canine Food Allergies. Can. Vet. J. 2019, 60, 268.
64. Gaschen, F. P.; Merchant, S. R. Adverse Food Reactions in Dogs and Cats. Vet. Clin. North Am. Small Anim. Pract. 2011, 41, 361-379, doi: 10.1016/J.CVSM.2011.02.005.
65. Verlinden, A.; Hesta, M.; Millet, S.; Janssens, G. P. J. Food Allergy in Dogs and Cats: A Review. Crit. Rev. Food Sci. Nutr. 2006, 46, 259-273, doi: 10.1080/10408390591001117.
66. Mueller, R. S.; Olivry, T.; Prelaud, P. Critically Appraised Topic on Adverse Food Reactions of Companion Animals (2): Common Food Allergen Sources in Dogs and Cats. BMC Vet. Res. 2016, 12, doi: 10.1186/s12917-016-0633-8.
67. Dodds, W. J. Diagnosis of Feline Food Sensitivity and Intolerance Using Saliva: 1000 Cases. Anim. an Open Access J. from MDP| 2019, 9, doi: 10.3390/ANI9080534.
68. Martín, A.; Sierra, M. P.; González, J. L.; Arevalo, M. A. Identification of Allergens Responsible for Canine Cutaneous Adverse Food Reactions to Lamb, Beef and Cow's Milk. Vet. Dermatol. 2004, 15, 349-356, doi: 10.1111/J.1365-3164.2004.00404.X.
69. Mullins, E.; Bresson, J. L.; Dalmay, T.; Dewhurst, I. C.; Epstein, M. M.; George Firbank, L.; Guerche, P.; Hejatko, J.; Naegeli, H.; Nogue, F.; et al. Scientific Opinion on Development Needs for the Allergenicity and Protein Safety Assessment of Food and Feed Products Derived from Biotechnology. EFSA J. 2022, 20, doi: 10.2903/j.efsa.2022.7044.
70. Halliwell, R.; Pucheu-Haston, C.M.; Olivry, T.; Prost, C.; Jackson, H.; Banovic, F.; Nuttall, T.; Santoro, D.; Bizikova, P.; Mueller, R.S. Feline Allergic Diseases: Introduction and Proposed Nomenclature. Vet. Dermatol. 2021, 32, 8-e2, doi: 10.1111/VDE.12899.
71. Sievers, F.; Wilm, A.; Dineen, D.; Gibson, T. J.; Karplus, K.; Li, W.; Lopez, R.; McWilliam, H.; Remmert, M.; Soding, J.; et al. Fast, Scalable Generation of High-Quality Protein Multiple Sequence Alignments Using Clustal Omega. Mol. Syst. Biol. 2011, 7, doi: 10.1038/msb.2011.75.
72. Waterhouse, A. M.; Procter, J. B.; Martin, D. M. A.; Clamp, M.; Barton, G. J. Jalview Version 2-A Multiple Sequence Alignment Editor and Analysis Workbench. Bioinformatics 2009, 25, 1189-1191, doi: 10.1093/bioinformatics/btp033.
73. Guex, N.; Peitsch, M.C. SWISS-MODEL and the Swiss-PdbViewer: An Environment for Comparative Protein Modeling. Electrophoresis 1997, 18, 2714-2723, doi: 10.1002/elps.1150181505.
74. Love, K. R.; Shah, K. A.; Whittaker, C. A.; Wu, J.; Bartlett, M. C.; Ma, D.; Leeson, R. L.; Priest, M.; Borowsky, J.; Young, S. K.; et al. Comparative Genomics and Transcriptomics of Pichia Pastoris. BMC Genomics 2016, 17, 1-17, doi: 10.1186/s12864-016-2876-y.
75. Cregg, J. M. Pichia Protocols; 2007; ISBN 9781588294296.
76. Pais-Chanfrau, J. M.; Trujillo-Toledo, L. E. Optimization of Culture Medium for Large-Scale Production of Heterologous Proteins in Pichia Pastoris to Be Used in Nanoscience and Other Biotechnological Fields. Recent Adv. Biol. Nanotechnol. 2016, 8, doi: 10.4172/0974-8369.1000279.

1. A method for preparing a pet food, wherein the method comprises adding an isolated myoglobin, wherein the isolated myoglobin is a rat myoglobin, a rabbit myoglobin, a mouse myoglobin, a mammoth myoglobin or a tuna myoglobin.

2. The method according to claim 1, wherein the isolated myoglobin is not comprised in a cell or a tissue before the addition.

3. The method for preparing a pet food according to claim 1,
wherein the isolated myoglobin is added as part of a composition, wherein the weight fraction of the isolated myoglobin in the protein fraction comprised in the composition is at least 75%.

4. The method for preparing a pet food according to claim 1, wherein the weight concentration of heme-proteins in the pet food is at least 1%, and wherein the weight fraction of myoglobin in the heme-proteins is at least 50%.

5. The A-method for preparing a pet food according to claim to claim 1, wherein the pet is a carnivore.

6. The A-method for preparing a pet food according to claim 1, wherein the pet is a dog or a cat.

7. The method for preparing a pet food according to claim 1, wherein the pet food is a dry pet food, a semi-moist pet food, a wet pet food, or any combination of these types of pet foods.

8. The A-method for preparing a pet food according to claim 1, wherein the pet food is a meat substitute.

9. The method for preparing a pet food according to claim 8, wherein the meat substitute is a cell-based meat substitute, a cultured meat substitute or a plant-based meat substitute.

10. The method for preparing a pet food according to claim 8, wherein the meat substitute mimics the aspect, form, structure, composition, palatability, flavor, texture, color, aroma, appearance and/or nutritional value of meat.

11. The method for preparing a pet food according to claim 10, wherein the meat substitute has an aroma characterized by a higher concentration of Mailard compounds when cooked, compared to a corresponding pet food which to which no isolated myoglobin has been added.

12. The method for preparing a pet food according to claim 1, wherein the isolated myoglobin is a recombinant myoglobin obtained from fermentation by a microorganism which has been genetically modified to express the recombinant myoglobin.

13. The method for preparing a pet food according to claim 12, wherein the microorganism is a bacterium, a yeast, a filamentous fungus or a cultured mammalian cell line, preferably Escherichia coli or Saccharomyces cerevisiae.

14. The method for preparing a pet food according to claim 1, wherein the isolated myoglobin is represented by one of the following amino acid sequences:
a) a sequence having at least 90% sequence identity with SEQ ID NO: 1, and having at least one of the following amino acid combinations:
M at position 10, and/or
A at position 36, and/or
S at position 58, and/or
M at position 10 and A at position 36, and/or
M at position 10 and S at position 58, and/or
A at position 36 and S at position 58, and/or
M at position 10 and A at position 36 and S at position 58;
b) a sequence having at least 90% sequence identity with SEQ ID NO: 2, and having an amino acid A at position 6;
c) a sequence having at least 90% sequence identity with SEQ ID NO: 3, and having at least one of the following amino acid combinations:
D at position 55, and/or
I at position 111, and/or
E at position 114, and/or
D at position 55 and I at position 111, and/or
D at position 55 and E at position 114, and/or
I at position 111 and E at position 114, and/or
D at position 55 and I at position 111 and E at position 114;
d) a sequence having at least 90% identity with SEQ ID NO: 4 or 5, and having F at position 30 and/or Q at position 65;
e) a sequence having at least 90% identity with SEQ ID NO: 6, and having H at position 65 and/or position 94;
f) a sequence having at least 90% identity with SEQ ID NO: 1, 2 or 3, and having at least one of the following amino acid combinations;
L at position 22, and/or
A or T at position 36, and/or
G at position 122;
L at position 22 and A or T at position 36, and/or
L at position 22 and G at position 122; and/or
A or T at position 36 and G at position 122, and/or
L at position 22 and A or T at position 36 and G at position 122;
g) a sequence having at least 90% identity with any one of SEQ ID NO: 1 to 5, and having I at position 87,
h) having at least 90% identity with SEQ ID NO: 1 or 3, and having at least one or more of the following amino acids or any combinations thereof;
N at position 49, and/or
E at position 54, and/or
C at position 67, and/or
T at position 75, and/or
Q at position 82, and/or
K at position 117, and/or
K at position 118, and/or
S at position 121,
i) having at least 90% identity with SEQ ID NO: 4,, and having at least one of the following amino acid combinations;
H at position 92, and/or
H at position 92 and F at position 30, and/or
H at position 92 and Q at position 65, and/or;
H at position 92 and F at position 30 and Q at position 65.

15. The method for preparing a pet food according to claim 1, wherein the isolated myoglobin is represented by any one of SEQ ID NO: 1 to 6, preferably by SEQ ID NO: 1 or 2.

16. A pet food obtainable by the method of claim 1.
